(19) United States
(12) Patent Application Publication
(10) Pub. No.: US 2008/0044378 A1
(43) Pub. Date: Feb. 21, 2008

(54) Methods and Compositions for Protein Production Using Adenoviral Vectors

(75) Inventors: Shuyuan Zhang, Sugar Land, TX (US); Hai Pham, Houston, TX (US); Ping Song, Houston, TX (US); Mingzhong Zheng, Houston, TX (US); Peter Clarke, Sugar Land, TX (US)

Correspondence Address: FULBRIGHT & JAWORSKI L.L.P., 600 CONGRESS AVE., SUITE 2400, AUSTIN, TX, 78701, US

(73) Assignee: INTROGEN THERAPEUTICS, INC., Austin, TX (US)

(21) Appl. No.: 11/748,826

(22) Filed: May 15, 2007

Related U.S. Application Data
(60) Provisional application No. 60/800,529, filed on May 15, 2006.

(51) Int. Cl. A61K 31/395; A61K 31/70; A61K 38/00; A61P 43/00; C12N 15/00

(52) U.S. Cl. 424/85.2; 424/130.1; 424/141.1; 424/94.63; 435/320.1; 435/69.1; 514/12; 514/44

(57) ABSTRACT

The present invention provides methods and compositions for recombinant protein production through replication-defective adenoviral vector infection of non-trans-complementation cell lines. Thus, this invention describes methods of heterologous protein production without an accompanied production of adenoviral vectors.

The present application claims benefit of priority to U.S. Provisional Application Ser. No. 60/800,529, filed May 15, 2006, the entire contents of which are hereby incorporated by reference.

### BACKGROUND OF THE INVENTION

1. Field of the Invention

The present invention relates generally to the fields of molecular biology and protein production. More particularly, it concerns methods and compositions for recombinant protein production through replication-defective adenoviral vector infection of non-trans-complementation cell lines. Thus, this invention describes methods of heterologous protein production without an accompanied production of adenoviral vectors.

2. Description of the Related Art

The expression of recombinant proteins in heterologous cells has been well documented. Such heterologous cell based systems for the production of recombinant proteins include prokaryotic cells, yeast, fungi, plant cells and mammalian cells. However, some heterologous cell based systems are not well suited for production of specific classes of proteins. For example, proteins that require post translational modification such as glycosylation cannot be produced in prokaryotic cell based systems.

Eukaryotic systems are therefore more suited in the production of eukaryotic derived proteins. Of the available eukaryotic cells for use in the field of recombinant protein production, mammalian cells are often a prime choice because of their ability to perform extensive post translational modifications. Accordingly, the expression of recombinant proteins in mammalian cells has become a routine technology in many cases.

Adenoviruses are currently the most commonly used vector for gene transfer in clinical settings. The vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). Several factors make adenoviral vectors particularly suitable for protein production, among these factors are: ease of manipulation of the adenoviral genome, lack of adenoviral genome rearrangement, the ability to replicate in an episomal manner without potential genotoxicity, and the ability to replace viral DNA with large sequences of foreign DNA for recombinant protein expression.

Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The E3 region is dispensable from the adenovirus genome (Jones and Shenk, 1978). The products of the late genes, including the majority of the viral capsid proteins, are expressed only after significant processing of a single primary transcript issued by the major late promoter (MLP). The MLP (located at 16.8 m.u.), is particularly efficient during the late phase of infection, and all the mRNA's issued from this promoter possess a 5′-tripartite leader (TPL) sequence which makes them preferred mRNA's for translation.

In nature, adenovirus can package approximately 105% of the wild-type genome (Ghosh-Choudhury et al., 1987), providing capacity for about 2 extra kb of DNA. Current replication-defective adenoviral vectors carry foreign DNA in either the E1, the E3 or both regions (Graham and Prevec, 1991). Accordingly, Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone. However, in order to produce adenovirus, replication-defective adenoviral vectors must be provided the functions of the E1 deleted region in trans, generally by a helper cell line.

Helper cell lines for adenoviral vector production may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293. A critical feature of helper cell lines is their ability to provide in trans, deleted E1 region of the adenovirus, or to provide proteins that will otherwise effectively substitute for this region so as to allow for effective adenoviral vector production.

Adenovirus vectors have been used in eukaryotic gene expression (Levrero et al., 1991; Gomez-Foix et al., 1992; Garnier et al., 1994). Garnier et al., for example, reported the use of an adenoviral vector system for the production of recombinant proteins in the E1 complimenting 293 cell line. When protein production is performed in an E1 complementing cell line, large amounts of adenovirus vector is produced together with the production of recombinant proteins. Production of the adenoviral vector however, would be expected to reduce the amount of recombinant proteins produced which may cause significant problems for downstream processing and purification of recombinant proteins, should the latter be the desired product. Accordingly, the use of an adenoviral vector system as a method of producing large amounts of recombinant proteins without the additional production of adenoviral particles may be desirable.

### SUMMARY OF THE INVENTION

The present invention provides a methods and compositions for producing exogenous proteins involving infecting a culture of host cells with an adenoviral vector encoding the exogenous protein and harvesting these proteins from the cell extract or supernatant. In particular, the invention concerns culturing cells with the vector to promote production of the exogenous protein(s), but not production of adenoviral particles.

“Exogenous” is defined herein to refer to any nucleic acid or protein that is not from or a host cell's genome. Therefore, the term “exogenous protein” refers to a protein that is not a gene product derived from the host cell's genome. The term “exogenous nucleic acid” refers to a nucleic acid sequence or molecule that is not part of the host cell's genomic DNA. Additionally, in certain embodiments, the exogenous nucleic acid or protein is a nucleic acid or protein that is not derived of the replication-defective adenoviral vector genome. However, in other embodiments, where production of an adenoviral protein is contemplated, such a protein may be rendered “exogenous” by the placement of its corresponding nucleic acid in a nucleic acid expression construct comprising a heterologous promoter and optionally a heterologous polyadenylation signal, and introduced in to a target cell.

It is also contemplated by methods of the present invention that in specific embodiments the nucleic acid expression construct of the adenoviral vector will comprise one or more promoter sequences. The promoter may or may not be heterologous. The term “heterologous” is used according to its ordinary and plain meaning to refer to a promoter that is not in nature associated with the particular coding sequence. In specific embodiments, the promoter is heterologous, while in other embodiments, the promoter is derived or is the promoter associated with the coding sequence for the exogenous nucleic acid.

The invention need not be limited to specific promoters or promoter embodiments. The heterologous promoter or promoters of the present invention may include any type of promoter. For example, the promoter may be a constitutive promoter, an inducible promoter, a repressible promoter, or a tissue selective promoter. A tissue selective promoter is defined herein to refer to any promoter which is relatively more active in certain tissue types compared to other tissue types. In certain embodiments of the viral vector of the present invention, the heterologous promoter or promoters is selected from the group of CMV IE promoter, RSV promoter, dectin-1 promoter, dectin-2 promoter, human CD11c promoter, mammalian F4/80 promoter, SM22 cc promoter, MHC class II promoter, hTERT promoter, CEA promoter, PSA promoter, probasin promoter, ARR2PB promoter, AFP promoter, SV40 early promoter, the U3 region of the Rous sarcoma virus, the U3 region other retroviruses, and any inducible promoter capable of operating in mammalian cells.

Further, it is contemplated by the methods of the present invention that the nucleic acid expression construct of the adenoviral vector will comprise one or more heterologous polyadenylation sequences, which refers to a polyadenylation sequence not associated in nature with the coding sequence in the nucleic acid construct. In certain embodiments of the viral vector of the present invention, the heterologous polyadenylation signal or signals is selected from the group of consisting of SV40 early polyadenylation signal, HSV TK polyadenylation signal, and human growth hormone polyadenylation signal. This list of polyadenylation signals is not intended to limit the invention.

In embodiments of the invention, the adenoviral vector is generated so that it is replication-defective and comprises a nucleic acid expression construct containing one or more nucleic acid sequences that encode one or more exogenous proteins. The culture of host cells, while capable of expressing one or more exogenous proteins encoded by the adenoviral vector, does not correct the replication defect by complementing the adenovirus vector for any mutations in genes required for replication. These cells would not be considered “helper cells” as that term has been applied in the context of virus production. Such host cells are defined herein as “non-trans-complementing.” Accordingly, in embodiments of the invention the adenovirus vector contains one or more mutations or deletions in its genome that render it replication-defective and the host cell does not contain any nucleic acid sequences that provide for trans-complementation of the adenovirus genomic replication defect.

In some embodiments, the replication defect of the adenoviral vector is due to a deletion in gene required for replication. In particular embodiments, the deletion is in the E1 region of the viral genome, which may be a deletion of or in the E1A and/or E1B region. In further embodiments, the deletion prevents the vector from expressing E1A and/or E1B with wild-type function. A number of such vectors exist in which the coding sequence for the E1 region has been mutated in some way, such as by deletion of all or part of it. In other embodiments, the E2 and/or E4 regions are partly or fully deleted alone or in conjunction with other mutations to prevent expression of proteins with wild-type function. In even further embodiments, the E3 region is also partly or fully deleted.

In specific embodiments of the present invention, the non-trans-complementing host cells are Vero, HeLa, Chinese hamster ovary, W138, BHK, COS-7, HepG2, RIN, MDCK, A549 or derivatives thereof. However, any cell line that is permissive for adenoviral infection that does not trans-complement the E1 deletion of adenoviral vectors may be used. In certain embodiments of the present invention, the non trans-complementing host cells are HeLa cells or derivatives thereof. A “derivative” cell refers to a cell or its progeny that was engineered from or became mutated with respect to a certain cell line. In certain embodiments, a derivative cell is one that has been engineered to contain one or more transgenes compared to the cell from which it was derived. In particular embodiments, the host cell is a primate cell, preferably a human cell.

Embodiments of the invention may involve variations in cell culturing or cell harvesting. In certain embodiments, cells are grown in serum-free media. This growth may be during an inoculum phase, during a cell growth phase (media is exchanged, during which media may or may not be collected to obtain protein), and/or during a protein production phase (phase during which media is not exchanged until media is collected for protein isolation). In certain embodiments, frozen cells are placed in serum-free media and do not contact serum thereafter. In other embodiments, frozen cells are initially placed in media containing serum, but when incubated in a volume that is about or at least about 5-, 10-, 20-, 50-, 100-fold or greater than the volume of media into which the frozen cells are placed, the cells are no longer in serum-containing media.

In additional embodiments, the cells are grown in a media in which animal-derived products have not been added. An animal-derived product refers to a product from an animal, and it includes, in some embodiments, bovine serum albumin, insulin, etc. In particular embodiments, cells are grown in a media lacking protein or a media in which protein has not been added. In particular embodiments, cells used for the invention are capable of growing in a serum-free and/or protein-free media.

The present invention also involves embodiments in which cells are grown for a certain number of generations or at least a certain number of generations. In some embodiments, cells are grown prior to or after transfection/infection for the following number of generations or at least the following number of generations: 5, 10, 15, 20, 25, 30, 35, 40, 45, 50, 60, 70, 80, 90, 100 or more generations, or any range derivable therein. In additional embodiments, cells are grown prior to or after transfection/infection for the following amount of time or at least the following amount of time: 1, 2, 3, 4, 5, 6, 7, 8, 9, 10, 11, 12, 13, 14, 15, 16, 17, 18, 19, 20, 21, 22, 23, 24 hours, and/or 1, 2, 3, 4, 5, 6, 7 days, and/or 1, 2, 3, 4, 5 weeks, and/or 1, 2, 3, 4, 5, 6 months, or any range derivable therein.

In further embodiments, cells are grown in a bioreactor in a volume of media that is about, at least about, or at most about 0.05, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 liters or more, or any range derivable therein. In some cases, cells are transferred to or kept in the same or larger volumes of media as time progresses. For example, cells may be placed in a 100 ml flask, and then transferred to a 1 liter bioreactor, and then to a 50 liter bioreactor before infection. Alternatively, cells may be kept in bags at some point.

Cells may be grown in media provided using batch or fed-batch, perfusion, or other exchange systems. Protein may be collected from media that is provided or collected in batch, fed-batch, perfused, chemostat cultured or otherwise exchanged.

In further embodiments, cells may be exposed to virus and then grown in media prior to protein harvesting for about, at least about, or at most about 12, 24, 36, 48, 60, 72, 84, 96, 108, 120, 132, 144, 156, 168, 180, 192, 204, 216, 228, 240 hours and/or 1, 2, 3, 4, 5, 6, 7, 8, 9, 10, 11, 12, 13, 14, 15, 16, 17, 18, 19, 20, 21 days, or any range derivable therein.

In many embodiments of the present invention, the harvested exogenous protein or proteins are subject to purification. Purification may involve a number of steps, for example, concentration and diafiltration by tangential flow ultrafiltration, chromatography or size resolution purification. In certain embodiments, chromatography is employed in heterologous protein purification. In specific embodiments, the chromatography is affinity chromatography or anion exchange chromatography. In still other embodiments heterologous protein purified by affinity chromatography is further subjected to anion-exchange chromatography. In some embodiments of the present invention, harvested heterologous protein is subjected to size resolution purification. In specific embodiments the size resolution purification involves a protein gel or size exclusion column. In certain embodiments of the methods of the present invention, the heterologous protein or proteins are placed in a pharmaceutically acceptable composition after purification.

It is also contemplated by the methods of the present invention that the gene or genes of the nucleic acid expression construct may be any gene or genes encoding an exogenous protein or proteins. In certain embodiments however, the genes are selected from the group of tumor suppressors, cytokines, pro-apoptotic factors antibodies and genes derived from microorganisms.

In certain embodiments, when the exogenous nucleic acid is a gene or genes encoding one or more tumor suppressors, any tumor suppressor gene or genes is contemplated. In specific embodiments, the tumor suppressor gene or genes are selected from the group consisting of APC, CYLD, HIN-1, KRAS2b, p16, p19, p21, p27, p27mt, p53, p57, p73, PTEN, MDA-7, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, ras, MMAC1, FCC, MCC, FUS1, Gene 26 (CACNA2D2), PL6, Beta* (BLU), Luca-1 (HYAL1), Luca-2 (HYAL2), 123F2 (RASSF1), 101F6, Gene 21 (NPRL2), or a gene encoding a SEM A3 polypeptide.

In still other embodiments, when the exogenous nucleic acid is a gene or genes encoding one or more cytokines, any gene encoding a cytokine is contemplated. In specific embodiments, the cytokine gene or genes is selected from the group consisting of GM-CSF, G-CSF, IL-1α, IL-10, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-25, IL-26, IL-27, IL-28, IL-29, IL-30, IL-31, IL-32 IFN-α, IFN-β, IFN-γ, MIP-1α, MIP-1β, TGF-β, TNF-α, TNF-β, PDGF, epidermal growth factor, keratinocyte growth factor, hepatycyte growth factor, TGF-α, TGF-β, VEGF and MDA-7.

In some embodiments, when the exogenous nucleic acid is a gene or genes encoding one or more pro-apoptotic factors, any gene or genes encoding such factors is contemplated. In specific embodiments, the pro-apoptotic factor gene or genes is selected from the group consisting of CD95, caspase-3, Bax, Bag-1, CRADD, TSSC3, bax, hid, Bak, MKP-7, PARP, bad, bcl-2, MST1, bbc3, Sax, BIK, and BID.

In certain embodiments, when the gene or genes of the nucleic acid expression construct are genes encoding antibodies, any gene or genes encoding such factors is contemplated. In specific embodiments, the antibody gene or genes is selected from the group consisting of cetuximab, rituximab, trastuzumab, gemtuzumab, alemtuzumab, ibritumomab, tositumomab, bevacizumab, alemtuzumab, HuPAM4, 3F8, G250, HuHMFG1, Hu3S193, hA20, SGN-30, RAV12, daclizumab, basiliximab, abciximab, palivizumab, infliximab, eculizumab, omalizumab, efalizumab, panitumumab and adalimumab.

In certain embodiments, the gene or genes of the exogenous nucleic acid are derived from microorganisms. While any gene derived from a microorganism is contemplated, in some embodiments the genes are derived from viruses, bacteria, fungi, or protozoa.

In specific embodiments, the microorganism from which the gene or genes are derived are viruses selected from the list of HIV-1, HIV-2, SIV, FIV, FeLV, Equine infectious anemia virus, eastern equine encephalitis virus, western equine encephalitis virus, Venezuelan equine encephalitis virus, rift valley fever virus, West Nile virus, yellow fever virus, Crimean-Congo hemorrhagic fever virus, dengue virus, SARS coronavirus, small pox virus, monkey pox virus, hepatitis A virus, hepatitis B virus, hepatitis C virus, influenza virus, adenovirus and rotavirus. In particular embodiments, the exogenous nucleic acid encodes an adenoviral gene, such as the adenovirus death protein gene (ADP).

In specific embodiments, the microorganism from which the gene or genes are derived are viruses selected from the list of Mycobacterium tuberculosis, Yersinia pestis, Rickettsia prowazekii, Rickettsia typhi, Rickettsia rickettsii, Ehrlichia chaffeensis, Francisella tularensis, Bacillus anthracis, Helicobacter pylori and Borrelia burgdorferi.

In specific embodiments, the microorganism from which the gene or genes are derived are viruses selected from the list of Plasmodium falciparum, Plasmodium vivax, Plasmodium ovate, Plasmodium malariae, and Giadaria intestinalis.

In specific embodiments, the microorganism from which the gene or genes are derived are viruses selected from the list of Histoplasma, Ciccidis, Immitis, Aspergillus, Actinomyces, Blastomyces, Candida and Streptomyces.

The methods of the present invention also involve culturing the non-trans-complementing cells. In specific embodiments, the culture of non trans-complementing host cells occurs in a bioreactor system, a microcarrier culture system, a multiplate culture system, a perfused packed bed reactor system, or a microencapsulation culture system.

In particular embodiments of the invention, the level of exogenous protein production is increased when a non-complementing cell line is employed, as compared to the level of protein production when a complementing cell line is employed. For instance, in certain embodiments, the production level of exogenous protein is expressed in terms of about, at least about, or at most about a 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 percent increase, or any range derivable therein, as compared to the production level in other cells transfected or infected with the relevant adenovirus vector. Alternatively, an increase in protein production levels may be expressed in terms of about, at least about, or at most about 2×, 3×, 4×, 5×, 10×, 15×, 20×, 25×, 30×, 35×, 40×, 45×, 50×, 60×, 70×, 80×, 90×, 100×, 110×, 120×, 130×, 140×, 150×, 160×, 170×, 180×, 190×, 200×, 210×, 220×, 230×, 240×, 250×, 260×, 270×, 280×, 290×, 300×, 310×, 320×, 330×, 340×, 350×, 360×, 370×, 380×, 390×, 400×, 410×, 420×, 430×, 440×, 450×, 460×, 470×, 480×, 490×, 500×, 600×, 700×, 800×, 900×, 1000× or more, or any range derivable therein, as compared to the production level in other cells transfected or infected with the relevant adenovirus vector.

Methods or compositions of the invention may involve or comprise an amount of produced protein. In some embodiments, the amount of protein produced and/or purified is about, at least about, or at most about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 600, 700, 800, 900, 1000 μg or mg, or any range or combination derivable therein. Thus, compositions of the invention include such amount of produced protein, which may or may not be purified to levels discussed above.

The embodiments in the Examples section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1—Western Blot showing MDA-7 production of Ad-mda-7 infected HeLa cells. Numbers represent hours post infection. Control was stably transfected 293M cells which expresses MDA-7 protein.

FIG. 2—Western Blot showing MDA-7 production of Ad-mda-7 infected HeLa cells and 293 cells. Control is stably transfected 293M cell which expresses MDA-7 protein. Culture media of HeLa cells from the wave bioreactor was harvested four days post infection and was subjected to centrifugation (WC) or filtration (WF). Culture media from 293M cells was harvested four days post infection. Culture media from 293 cells was harvested two to six days post infection (D2, D3, D4, D5 and D6).

FIG. 3—Chromatogram of Phenyl-Sepharose FF column purification 3 ml/min loading rate. Sample collection took place at 10 ml intervals during the linear curve as indicated by numbers 1-16.

FIG. 4—Chromatogram of Butyl-Sepharose FF column purification, 3 ml/min loading rate. Sample collection took place at 10 ml intervals during the linear curve as indicated by numbers 1-15.

FIG. 5—Chromatogram of Hydroxyapatite Type 1 column purification, 3 ml/min loading rate. Sample material was previously purified via Butyl-Sepharose FF column. Sample collection took place at 10 ml intervals during the linear curve as indicated by numbers 1-6.

FIG. 6—Chromatogram of Butyl-Sepharose column purification, 3 ml/min loading rate. Sample material was previously purified via Phenyl-Sepharose FF column.

FIG. 7A—SDS-PAGE of fractions purified by Phenyl-sepharose column. Numbers correspond to collected fractions. Arrow indicates expected size of Mda-7 protein.

FIG. 7B—Western blot of fractions purified by Phenyl-Sepharose FF column showing the presence of MDA-7. Numbers correspond to collected fractions.

FIG. 8—SDS-PAGE of mda-7 samples subject to Phenyl-Sepharose FF column purification, Butyl-Sepharose FF column purification, or a combination. Lanes 2, 4, 6 and 7 represent recombinant MDA-7 protein as a control. Lane 3 represents the MDA-7 fraction eluted from the Phenyl-Sepharose FF Column, fraction 8. Lane 5 represents the MDA-7 fraction eluted from the Butyl-Sepharose FF column, fraction 10. Lane 8 represents the fraction eluted from the combination of Phenyl- and Butyl-sepharose FF columns.

FIG. 9A—SDS-PAGE analysis of MDA-7 samples subject to Butyl-Sepharose FF column purification. FT—flow through fraction, MW—molecular weight marker.

FIG. 9B—Western Blot of fractions purified by Butyl-Sepharose FF column showing the presence of MDA-7. Numbers correspond to collected fractions.

FIG. 10A—SDS-PAGE analysis of MDA-7 samples subject to Butyl-Sepharose FF followed by Hydroxyapatite Type 1 column purification. Arrow corresponds to MDA-7 protein

FIG. 10B—Western blot of fractions purified by Butyl-Sepharose FF followed by Hydroxyapatite Type 1 column purification. Arrow corresponds to MDA-7 protein.

FIG. 11—Optimization of HeLa cell infection conditions for production of MDA-7 protein. Levels of MDA-7 protein in the culture media increased as the virus infection proceeded. The highest levels of MDA-7 protein were observed after 6 days post infection when HeLa cells were infected with Ad-mda7 at a MOI of 3000 vp/cell.

FIG. 12—Comparison of tumor cell killing of supernatants from Ad-mda7 infected HeLa cells. Levels of MDA-7 protein in the culture media resulted in dramatic increase in cell death of both MeWo and MDA-MB-543 cell lines as compared to supernatant from HeLa cells which were not infected with Ad-mda7. Also observed was the fact that the percentage of cell death was dose dependant, with increasing dosages of supernatant containing MDA-7 protein resulting in greater levels of target cell death.

### DESCRIPTION OF THE ILLUSTRATIVE EMBODIMENTS

It has been shown that adenoviral vectors can successfully be used for gene therapy. Successful studies in administering recombinant adenovirus to different tissues have proven the effectiveness of adenoviral vectors in therapy. In addition to their ability to transduce a wide variety of cell types, adenoviral vectors are adept in eukaryotic gene expression. This success has lead to the use of such vectors in human trials. The properties of adenoviral vectors in eukaryotic gene expression make these vectors promising tools in the development of recombinant protein production. In certain embodiments, the present invention provides methods for the production of large amounts of recombinant proteins without corresponding adenoviral particle production using these vectors.

In certain embodiments, present invention involves a novel method of rapid production of proteins. The production process is based on the infection of non-trans-complementing protein producer cells with an adenoviral vector comprising a nucleic acid encoding a heterologous protein of interest. In this invention, a replication-defective adenoviral vector encoding a gene of a heterologous protein is used to infect an a non trans-complementing cell line grown in media. Preferably, the replication-defective adenoviral vector contains a deletion of the E1 region. Optionally, the replication-defective adenoviral vector may contain other deletions, such as deletions in the E3 or E4 region of the adenoviral genome. Because of the lack of trans-complementing adenoviral genes in the cell line (such as the E1 gene in the case of an E1-deleted adenoviral vector), no further amplification of the infected adenoviral vector is expected. Because of the high infection efficiency of the adenoviral vector, high levels of the heterologous protein are produced from the infected cells. Since it is relatively easy to construct a replication-defective adenoviral vector, such as an E1-deleted adenoviral vector, following standard procedure, this novel method can greatly simplify the production of protein products, such as therapeutic recombinant proteins and monoclonal antibodies. Since the production is produced in a human cell line, the protein product will have the desired glycosylation form without the need for re-folding and humanization. Additionally, the methods of the present invention are expected to be useful for the production of cytotoxic protein products where toxicity would make the construction of stable producer cells difficult or impossible.

### I. NUCLEIC ACIDS

In the embodiments of the present invention, the methods of protein production set forth herein include an adenoviral vector with a heterologous nucleic acid sequence comprising one or more genes. For example, the gene or genes may be a therapeutic gene, such as a tumor suppressor gene, a pro-apoptotic gene, a gene that encodes a cytokine or a gene that encodes an antibody or a gene that encodes an antigen of a heterologous microorganism. Any gene or genes known to those of ordinary skill in the art is contemplated for inclusion in the methods of the present invention. Particular genes that are contemplated are those that considered to be of use in the detection or prevention or treatment of a disease in a subject. The term “gene” is used to refer to a nucleic acid sequence that encodes a functional protein, polypeptide, or peptide-encoding unit.

In certain embodiments of the present invention, a therapeutic gene is encoded by a nucleic acid. A “therapeutic gene” is a gene which can be administered to a subject for the purpose of treating or preventing a disease. For example, a therapeutic gene can be a gene administered to a subject for treatment or prevention of a hyperproliferative disease, such as cancer. Tumor suppressor genes, pro-apoptotic genes, and genes encoding cytokines are exemplary genes that can be applied in the treatment of a hyperproliferative disease, and are discussed in greater detail below.

Examples of therapeutic genes include, but are not limited to, Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, IL-13, GM-CSF, G-CSF, thymidine kinase, mda-7, fus-1, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, Rb, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, or MCC.

Other examples of therapeutic genes include genes encoding enzymes. Examples include, but are not limited to, ACP desaturase, an ACP hydroxylase, an ADP-glucose pyrophorylase, an ATPase, an alcohol dehydrogenase, an amylase, an amyloglucosidase, a catalase, a cellulase, a cyclooxygenase, a decarboxylase, a dextrinase, an esterase, a DNA polymerase, an RNA polymerase, a hyaluron synthase, a galactosidase, a glucanase, a glucose oxidase, a GTPase, a helicase, a hemicellulase, a hyaluronidase, an integrase, an invertase, an isomerase, a kinase, a lactase, a lipase, a lipoxygenase, a lyase, a lysozyme, a pectinesterase, a peroxidase, a phosphatase, a phospholipase, a phosphorylase, a polygalacturonase, a proteinase, a peptidease, a pullanase, a recombinase, a reverse transcriptase, a topoisomerase, a xylanase, a reporter gene, an interleukin, or a cytokine.

Further examples of therapeutic genes include the gene encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione beta.-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, beta.-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase, galactose-1-phosphate uridyltransferase, phenylalanine hydroxylase, glucocerbrosidase, sphingomyelinase, α-L-iduronidase, glucose-6-phosphate dehydrogenase, glucosyltransferase; HSV thymidine kinase, or human thymidine kinase.

Therapeutic genes also include genes encoding hormones. Examples include, but are not limited to, genes encoding growth hormone, prolactin, placental lactogen, luteinizing hormone, follicle-stimulating hormone, chorionic gonadotropin, thyroid-stimulating hormone, leptin, adrenocorticotropin, angiotensin I, angiotensin II, β-endorphin, β-melanocyte stimulating hormone, cholecystokinin, endothelin I, galanin, gastric inhibitory peptide, glucagon, insulin, lipotropins, neurophysins, somatostatin, calcitonin, calcitonin gene related peptide, β-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, alpha melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, or thyrotropin releasing hormone.

Other examples of therapeutic genes include genes encoding antigens present in pathogens, or immune effectors involved in autoimmunity. These genes can be applied, for example, in formulations that would be applied in vaccinations for immune therapy or immune prophylaxis of infectious diseases and autoimmune diseases.

As will be understood by those in the art, the term “therapeutic gene” includes genomic sequences, cDNA sequences, and smaller engineered gene segments that express, or may be adapted to express, proteins, polypeptides, domains, peptides, fusion proteins, and mutants. The nucleic acid molecule encoding a therapeutic gene may comprise a contiguous nucleic acid sequence of about 5 to about 12000 or more nucleotides, nucleosides, or base pairs.

Encompassed within the definition of “therapeutic gene” is a “biologically functional equivalent” therapeutic gene. Accordingly, sequences that have about 70% to about 99% homology of amino acids that are identical or functionally equivalent to the amino acids of the therapeutic gene will be sequences that are biologically functional equivalents provided the biological activity of the protein is maintained.

A. Nucleic Acids Encoding Tumor Suppressors

The phrase “nucleic acid sequence encoding,” as set forth throughout this application, refers to a nucleic acid which directs the expression of a specific protein or peptide. The nucleic acid sequences include both the DNA strand sequence that is transcribed into RNA and the RNA sequence that is translated into protein.

A “tumor suppressor amino acid sequence” refers to a polypeptide that, when present in a cell, reduces the tumorigenicity, malignancy, or hyperproliferative phenotype of the cell. The nucleic acid sequences encoding tumor suppressor amino acid sequences include both the full length nucleic acid sequence of the tumor suppressor gene, as well as non-full length sequences of any length derived from the full length sequences. It being further understood that the sequence includes the degenerate codons of the native sequence or sequences which may be introduced to provide codon preference in a specific host cell.

“Tumor suppressor genes” are generally defined herein to refer to nucleic acid sequences that reduce the tumorigenicity, malignancy, or hyperproliferative phenotype of the cell. Thus, the absence, mutation, or disruption of normal expression of a tumor suppressor gene in an otherwise healthy cell increases the likelihood of, or results in, the cell attaining a neoplastic state. Conversely, when a functional tumor suppressor gene or protein is present in a cell, its presence suppresses the tumorigenicity, malignancy or hyperproliferative phenotype of the host cell. Examples of tumor suppressor nucleic acids within this definition include, but are not limited to APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, scFV, ras, MMAC1, FCC, MCC, Gene 26 (CACNA2D2), PL6, Beta* (BLU), Luca-1 (HYAL1), Luca-2 (HYAL2), 123F2 (RASSF1), 101F6, Gene 21 (NPRL2), or a gene encoding a SEM A3 polypeptide and FUS1. Other exemplary tumor suppressor genes are described in a database of tumor suppressor genes at www.cise.ufl.edu/˜yy1/HTML-TSGDB/Homepage.html. This database is herein specifically incorporated by reference into this and all other sections of the present application. Nucleic acids encoding tumor suppressor genes, as discussed above, include tumor suppressor genes, or nucleic acids derived therefrom (e.g., cDNAs, cRNAs, mRNAs, and subsequences thereof encoding active fragments of the respective tumor suppressor amino acid sequences), as well as vectors comprising these sequences. One of ordinary skill in the art would be familiar with tumor suppressor genes that can be applied in the present invention.

One of the best known tumor suppressor genes is p53. p53 is central to many of the cell's anti-cancer mechanisms. It can induce growth arrest, apoptosis and cell senescence. In normal cells p53 is usually inactive, bound to the protein MDM-2, which prevents its action and promotes its degradation. Active p53 is induced after the effects of various cancer-causing agents such as UV radiation, oncogenes and some DNA-damaging drugs. DNA damage is sensed by ‘checkpoints’ in a cell's cycle, and causes proteins such as ATM, Chk1 and Chk2 to phosphorylate p53 at sites that are close to the MDM2-binding region of the protein. Oncogenes also stimulate p53 activation, mediated by the protein p14ARF. Some oncogenes can also stimulate the transcription of proteins which bind to MDM2 and inhibit its activity. Once activated p53 has many anticancer mechanisms, the best documented being its ability to bind to regions of DNA and activate the transcription of genes important in cell cycle inhibition, apoptosis, genetic stability, and inhibition of angiogenesis (Vogelstein et al, 2000). Studies have linked the p53 and pRB tumour suppressor pathways, via the protein p14ARF, raising the possibility that the pathways may regulate each other (Bates et al, 1998).

B. Nucleic Acids Encoding Pro-Apoptotic Proteins

Pro-apoptotic genes encode proteins that induce or sustain apoptosis to an active form. The present invention contemplates inclusion of any pro-apoptotic amino acid sequence known to those of ordinary skill in the art. Exemplary pro-apoptotic genes include CD95, caspase-3, Bax, Bag-1, CRADD, TSSC3, bax, hid, Bak, MKP-7, PERP, bad, bcl-2, MST1, bbc3, Sax, BIK, and BID. One of ordinary skill in the art would be familiar with pro-apoptotic genes, and other such genes not specifically set forth herein that can be applied in the methods and compositions of the present invention.

Nucleic acids encoding pro-apoptotic amino acid sequences include pro-apoptotic genes or nucleic acids derived there from (e.g., cDNAs, cRNAs, mRNAs, and subsequences thereof encoding active fragments of the respective pro-apoptotic amino acid sequence), as well as vectors comprising these sequences. A “pro-apoptotic amino acid sequence” refers to a polypeptide that, when present in a cell, induces or promotes apoptosis.

C. Nucleic Acids Encoding Cytokines

The term “cytokine” is a generic term for proteins released by one cell population which act on another cell as intercellular mediators. A “cytokine amino acid sequence” refers to a polypeptide that, when present in a cell, maintains some or all of the function of a cytokine. The nucleic acid sequences encoding cytokine amino acid sequences include both the full length nucleic acid sequence of the cytokine, as well as non-full length sequences of any length derived from the full length sequences. It being further understood, as discussed above, that the sequence includes the degenerate codons of the native sequence or sequences which may be introduced to provide codon preference in a specific host cell.

Examples of such cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); prostaglandin, fibroblast growth factors (FGFs) such as FGF-A and FGF-β; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1-α, IL-2, IL-3, IL-4, IL-5, IL-6, IL-7, IL-8, IL-9, IL-11, IL-12; IL-13, IL-14, IL-15, IL-16, IL-17, IL-18, IL-22, LIF, G-CSF, GM-CSF, M-CSF, EPO, kit-ligand, mda-7 or FLT-3.

A non limiting example of growth factor cytokines involved in wound healing include: epidermal growth factor, platelet-derived growth factor, keratinocyte growth factor, hepatycyte growth factor, transforming growth factors (TGFs) such as TGF-α and TGF-β, and vascular endothelial growth factor (VEGF). These growth factors trigger mitogenic, motogenic and survival pathways utilizing Ras, MAPK, PI-3K/Akt, PLC-gamma and Rho/Rac/actin signaling. Hypoxia activates pro-angiogenic genes (e.g., VEGF, angiopoietins) via HIF, while serum response factor (SRF) is critical for VEGF-induced angiogenesis, re-epithelialization and muscle restoration. EGF, its receptor, HGF and Cox2 are important for epithelial cell proliferation, migration re-epithelializaton and reconstruction of gastric glands. VEGF, angiopoietins, nitric oxide, endothelin and metalloproteinases are important for angiogenesis, vascular remodeling and mucosal regeneration within ulcers. (Tamawski, 2005)

Another example of a cytokine is IL-10. IL-10 is a pleiotropic homodimeric cytokine produced by immune system cells, as well as some tumor cells (Ekmekcioglu et al., 1999). Its immunosuppressive function includes potent inhibition of proinflammatory cytokine synthesis, including that of IFNγ, TNFα, and IL-6 (De Waal Malefyt et al., 1991). The family of IL-10-like cytokines is encoded in a small 195 kb gene cluster on chromosome 1q32, and consists of a number of cellular proteins (IL-10, IL-19, IL-20, MDA-7) with structural and sequence homology to IL-10 (Kotenko et al., 2000; Gallagher et al., 2000; Blumberg et al., 2001; Dumoutier et al., 2000; Knapp et al., 2000; Jiang et al., 1995a; Jiang et al., 1996).

A recently discovered putative member of the cytokine family is mda-7. The MDA-7 protein has been characterized as an IL-10 family member and is also known as IL-24. Chromosomal location, transcriptional regulation, murine and rat homologue expression, and putative protein structure all allude to MDA-7 being a cytokine (Knapp et al., 2000; Schaefer et al., 2000; Soo et al., 1999; Zhang et al., 2000). Similar to GM-CSF, TNFα, and IFNγ transcripts, all of which contain AU-rich elements in their 3′UTR targeting mRNA for rapid degradation, MDA-7 has three AREs in its 3′UTR¹⁷. Mda-7 mRNA has been identified in human PBMC (Ekmekcioglu, et al., 2001), and although no cytokine function of human MDA-7 protein has been previously reported, MDA-7 has been designated as IL-24 based on the gene and protein sequence characteristics (NCBI database accession XM_{—}001405).

D. Nucleic Acids Encoding Proteins of Microorganisms

The term microorganism includes viruses, bacteria, microscopic fungi, protazoa and other microscopic parasites. A “microorganism antigen amino acid sequence” refers to a polypeptide that, when presented on the cell surface by antigen presenting cells (APCs), induces an immune response.

Examples of viruses from which microorganism amino acid sequences may be derived include: human herpes viruses (HHVs)-1 through 8; herpes B virus; HPV-16, 18, 31, 33, and 45; hepatitis viruses A, B, C, 6; poliovirus; rotavirus; influenza; lentiviruses; HTLV-1; HTLV-2; equine infectious anemia virus; eastern equine encephalitis virus; western equine encephalitis virus; Venezuelan equine encephalitis virus; rift valley fever virus; West Nile virus; yellow fever virus; Crimean-Congo hemorrhagic fever virus; dengue virus; SARS coronavirus; small pox virus; monkey pox virus, and/or the like.

Examples of bacteria from which microorganism antigen amino acid sequences may be derived include: Mycobacterium tuberculosis; Yersinia pestis; Rickettsia prowazekii; Rickettsia typhi; Rickettsia rickettsii; Ehrlichia chaffeensis; Francisella tularensis; Bacillus anthracis; Helicobacter pylori; Salmonella typhi; Borrelia burgdorferi; Streptococcus mutans; and/or the like.

Examples of fungi from which microorganism antigen amino acid sequences may be derived include: Histoplasma; Ciccidis; Immitis; Aspergillus; Actinomyces; Blastomyces; Candida, Streptomyces and/or the like.

Examples of protazoa or other microorganisms from which antigen amino acid sequences may be derived include: Plasmodium falciparum, Plasmodium vivax; Plasmodium ovale; Plasmodium malariae; Giadaria intestinalis and/or the like.

E. Nucleic Acids Encoding Antibodies

The term “antibody” is a generic term for a protein produced by B cells or B cell hybridomas designed to bind to and neutralize antigens, such as antigens derived from bacteria, viruses, or cell surface proteins. An “antibody amino acid sequence” refers to a polypeptide that, when present in a cell, maintains some or all of the function of a antibody. The nucleic acid sequences encoding antibody amino acid sequences include both the full length nucleic acid sequence of the cytokine, as well as non-full length sequences of any length derived from the full length sequences. It being further understood, as discussed above, that the sequence includes the degenerate codons of the native sequence or sequences which may be introduced to provide codon preference in a specific host cell. Table 1 lists antibodies contemplated for clinical applications and their targets.

TABLE 1

| Generic Name | Target |
|---|---|
| cetuximab | EGFR |
| panitumumab | EGFR |
| trastuzumab | erbB2 receptor |
| bevacizumab | VEGF |
| alemtuzumab | CD52 |
| gemtuzumab ozogamicin | CD33 |
| rituximab | CD20 |
| tositumomab | CD20 |
| matuzumab | EGFR |
| ibritumomab tiuxetan | CD20 |
| tositumomab | CD20 |
| HuPAM4 | MUC1 |
| MORAb-009 | mesothelin |
| G250 | carbonic anhydrase IX |
| mAb 8H9 | 8H9 antigen |
| M195 | CD33 |
| ipilimumab | CTLA4 |
| HuLuc63 | CS1 |
| alemtuzumab | CD53 |
| epratuzumab | CD22 |
| BC8 | CD45 |
| HuJ591 | Prostate specific membrane antigen |
| hA20 | CD20 |
| lexatumumab | TRAIL receptor-2 |
| pertuzumab | HER-2 receptor |
| Mik-beta-1 | IL-2R |
| RAV12 | RAAG12 |
| SGN-30 | CD30 |
| AME-133v | CD20 |
| HeFi-1 | CD30 |
| BMS-663513 | CD137 |
| volociximab | anti-α5β1 integrin |
| GC1008 | TGFβ |
| HCD122 | CD40 |
| siplizumab | CD2 |
| MORAb-003 | folate receptor alpha |
| CNTO 328 | IL-6 |
| MDX-060 | CD30 |
| ofatumumab | CD20 |
| SGN-33 | CD33 |

### II. EXPRESSION CASSETTES

A. Overview

In certain embodiments of the present invention, the methods set forth herein involve nucleic acid sequences wherein the nucleic acid is comprised in an “expression cassette.” Throughout this application, the term “expression cassette” is meant to include any type of genetic construct containing a nucleic acid coding for a gene product in which part or all of the nucleic acid encoding sequence is capable of being transcribed.

B. Promoters and Enhancers

In order for the expression cassette to effect expression of a transcript, the nucleic acid encoding gene will be under the transcriptional control of a promoter. A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

Any promoter known to those of ordinary skill in the art that would be active in a cell in any cell in a subject is contemplated as a promoter that can be applied in the methods and compositions of the present invention. As discussed elsewhere, a subject can be any subject, including a human and another mammal, such as a mouse or laboratory animal. One of ordinary skill in the art would be familiar with the numerous types of promoters that can be applied in the present methods and compositions. In certain embodiments, for example, the promoter is a constitutive promoter, an inducible promoter, or a repressible promoter. The promoter can also be a tissue selective promoter. A tissue selective promoter is defined herein to refer to any promoter which is relatively more active in certain tissue types compared to other tissue types. Thus, for example, a liver-specific promoter would be a promoter which is more active in liver compared to other tissues in the body. One type of tissue-selective promoter is a tumor selective promoter. A tumor selective promoter is defined herein to refer to a promoter which is more active in tumor tissue compared to other tissue types. There may be some function in other tissue types, but the promoter is relatively more active in tumor tissue compared to other tissue types. Examples of tumor selective promoters include the hTERT promoter, the CEA promoter, the PSA promoter, the probasin promoter, the ARR2PB promoter, and the AFP promoter.

The promoter will be one which is active in the target cell. For instance, where the target cell is a keratinocyte, the promoter will be one which has activity in a keratinocyte. Similarly, where the cell is an epithelial cell, skin cell, mucosal cell or any other cell that can undergo transformation by a papillomavirus, the promoter used in the embodiment will be one which has activity in that particular cell type.

A promoter may be one naturally associated with a gene or sequence, as may be obtained by isolating the 5′-non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202 and U.S. Pat. No. 5,928,906, each incorporated herein by reference). Furthermore, it is contemplated the control sequences that direct transcription and/or expression of sequences within non-nuclear organelles such as mitochondria, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. 2001, incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The particular promoter that is employed to control the expression of the nucleic acid of interest is not believed to be critical, so long as it is capable of expressing the polynucleotide in the targeted cell at sufficient levels. Thus, where a human cell is targeted, it is preferable to position the polynucleotide coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter and the Rous sarcoma virus long terminal repeat can be used. The use of other viral or mammalian cellular or bacterial phage promoters which are well-known in the art to achieve expression of polynucleotides is contemplated as well, provided that the levels of expression are sufficient to produce a growth inhibitory effect.

By employing a promoter with well-known properties, the level and pattern of expression of a polynucleotide following transfection can be optimized. For example, selection of a promoter which is active in specific cells, such as tyrosine (melanoma), alpha-fetoprotein and albumin (liver tumors), CC10 (lung tumors) and prostate-specific antigen (prostate tumor) will permit tissue-specific expression of the therapeutic nucleic acids set forth herein. Table 2 lists additional examples of promoters/elements which may be employed, in the context of the present invention, to regulate the expression of the anti-cancer genes. This list is not intended to be exhaustive of all the possible promoter and enhancer elements, but, merely, to be exemplary thereof.

TABLE 2

| Promoter/Enhancer | References |
|---|---|
| Immunoglobulin Heavy Chain | Banerji et al., 1983; Grilles et al., 1983; Grosschedl |
| | et al., 1985; Atchinson et al., 1986, 1987; Imler et |
| | al., 1987; Weinberger et al., 1984; Kiledjian et al., |
| | 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo et |
| | al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et |
| | al., 1989 |
| Prealbumin (Transthyretin) | Costa et al., 1988 |
| Elastase I | Omitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypsin | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; Imbra |
| | et al., 1986; Kadesch et al., 1986; Wang et al., |
| | 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen et |
| | al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky et |
| | al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe et |
| | al., 1988; Rosen et al., 1988; Berkhout et al., 1989; |
| | Laspia et al., 1989; Sharp et al., 1989; Braddock et |
| | al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and continguous, often seeming to have very similar modular organization.

Additionally, any promoter/enhancer combination (as per the Eukaryotic Promoter Data Base EPDB) could also be used to drive expression of a gene. Use of a T3, T7, or SP6 cytoplasmic expression system is another possible embodiment. Eukaryotic cells can support cytoplasmic transcription from certain bacteriophage promoters if the appropriate bacteriophage polymerase is provided, either as part of the delivery complex or as an additional expression vector.

Further selection of a promoter that is regulated in response to specific physiologic signals can permit inducible expression of a construct. For example, with the polynucleotide under the control of the human PAI-1 promoter, expression is inducible by tumor necrosis factor. Table 3 provides examples of inducible elements, which are regions of a nucleic acid sequence that can be activated in response to a specific stimulus.

TABLE 3

| Element | Inducer | References |
|---|---|---|
| MT II | Phorbol Ester | Palmiter et al., 1982; |
| | (TFA) | Haslinger et al., 1985; |
| | Heavy metals | Searle et al., 1985; Stuart et |
| | | al., 1985; Imagawa et al., |
| | | 1987, Karin et al., 1987; |
| | | Angel et al., 1987b; |
| | | McNeall et al., 1989 |
| MMTV (mouse | Glucocor- | Huang et al., 1981; Lee et |
| mammary | ticoids | al., 1981; Majors et al., |
| tumor virus) | | 1983; Chandler et al., 1983; |
| | | Ponta et al., 1985; Sakai et |
| | | al., 1988 |
| β-Interferon | poly(rI)x | Tavernier et al., 1983 |
| | poly(rc) | |
| Adenovirus 5 E2 | E1A | Imperiale et al., 1984 |
| Collagenase | Phorbol Ester | Angel et al., 1987a |
| | (TPA) | |
| Stromelysin | Phorbol Ester | Angel et al., 1987b |
| | (TPA) | |
| SV40 | Phorbol Ester | Angel et al., 1987b |
| | (TPA) | |
| Murine MX Gene | Interferon, | Hug et al., 1988 |
| | Newcastle | |
| | Disease Virus | |
| GRP78 Gene | A23187 | Resendez et al., 1988 |
| α-2-Macro- | IL-6 | Kunz et al., 1989 |
| globulin | | |
| Vimentin | Serum | Rittling et al., 1989 |
| MHC Class I | Interferon | Blanar et al., 1989 |
| Gene H-2κb | | |
| HSP70 | E1A, SV40 | Taylor et al., 1989, 1990a, |
| | Large T | 1990b |
| | Antigen | |
| Proliferin | Phorbol | Mordacq et al., 1989 |
| | Ester-TPA | |
| Tumor Necrosis | PMA | Hensel et al., 1989 |
| Factor | | |
| Thyroid Stimu- | Thyroid | Chatterjee et al., 1989 |
| lating Hormone | Hormone | |
| α Gene | | |

C. Initiation Signals

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

D. IRES

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819). One of ordinary skill in the art would be familiar with the application of IRES in gene therapy.

E. Multiple Cloning Sites

Expression cassettes can include a multiple cloning site (MCS), which is a nucleic acid region that contains multiple restriction enzyme sites, any of which can be used in conjunction with standard recombinant technology to digest the vector. See Carbonelli et al. (1999); Levenson et al. (1998); Cocea (1997). “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see Chandler et al., 1997).

F. Polyadenylation Signals

In expression, one will typically include a polyadenylation signal to effect proper polyadenylation of the transcript. The nature of the polyadenylation signal is not believed to be crucial to the successful practice of the invention, and/or any such sequence may be employed. Particular embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

G. Other Expression Cassette Components

In certain embodiments of the invention, cells infected by the adenoviral vector may be identified in vitro by including a reporter gene in the expression vector. Such reporter genes would confer an identifiable change to the cell permitting easy identification of cells containing the expression vector. Generally, a selectable reporter is one that confers a property that allows for selection. A positive selectable reporter is one in which the presence of the reporter gene allows for its selection, while a negative selectable reporter is one in which its presence prevents its selection. An example of a positive selectable marker is a drug resistance marker.

Usually the inclusion of a drug selection marker aids in the cloning and identification of infected cells, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of reporters including screenable reporters such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic reporters, possibly in conjunction with FACS analysis. The marker used is not believed to be important, so long as it is capable of being expressed simultaneously with the nucleic acid encoding a gene product. Further examples of selectable and screenable reporters are well known to one of skill in the art.

### III. HOST CELLS

A. Cells

In a particular embodiment, the generation of heterologous proteins derived from replication-defective adenoviral vectors depends on the use of non trans-complementing cell lines. In contrast, a defective adenoviral vector helper cell line, such as the 293 cell line, constitutively expresses E1 proteins (Graham et al., 1977), and therefore compliments the E1 deletion of the defective adenoviral vector, thereby allowing for production of virus.

A first aspect of the present invention is the non trans-complementing which do not express parts of the adenoviral genome. Selected cell lines of the present invention are not capable of supporting replication of adenoviral vectors having defects in certain adenoviral genes necessary for viral replication.

Non-trans-complementing cells according to the present invention are derived from a mammalian cell, such as a primate cell. Although various primate cells are contemplated, in particular human cells are contemplated, although any type of cell that is capable of supporting heterologous gene expression from a replication-defective adenoviral vector would be acceptable in the practice of the invention. In one embodiment, HeLa cells, a human cervical cancer cell line transformed with human papilloma virus subtype 18, is contemplated. Other cell types might include, but are not limited to Vero cells, HeLa derived cell lines and cell lines of Chinese hamster ovary, W138, BHK, COS-7, HepG2, 3T3, RIN, MDCK and A549, as long as the cells are adenovirus permissive. The term “adenovirus permissive” means that a replication-defective adenoviral vector of the present invention would be able to infect the cell and produce RNA transcripts and subsequent protein derived from the exogenous gene associated with the adenoviral vector.

B. Growth in Selection Media

In certain embodiments, it may be useful to employ selection systems that preclude growth of undesirable cells. This may be accomplished by virtue of permanently transforming a cell line with a selectable marker or by transducing or infecting a cell line with a viral vector that encodes a selectable marker. In either situation, culture of the transformed/transduced cell with an appropriate drug or selective compound will result in the enhancement, in the cell population, of those cells carrying the marker.

Examples of markers include, but are not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to methotrexate; gpt, that confers resistance to mycophenolic acid; neo, that confers resistance to the aminoglycoside G418; and hygro, that confers resistance to hygromycin.

C. Growth During Weaning

Serum weaning adaptation of anchorage-dependent cells into serum-free suspension cultures have been used for the production of recombinant proteins (Berg, 1993) and viral vaccines (Perrin, 1995). Gilbert reported the adaptation of 293A cells into serum-free suspension cultures for adenovirus and recombinant protein production (Gilbert, 1996). A similar adaptation method had been used for the adaptation of A549 cells into serum-free suspension culture for adenovirus production (Morris et al., 1996). Cell-specific virus yields in the adapted suspension cells, however, are about 5-10-fold lower than those achieved in the parental attached cells.

D. Adaptation of Cells for Suspension Culture

Various methods have been used to adapt cells into suspension cultures. For example, in the present invention, HeLa cells adapted for growth in serum-free conditions were adapted into a suspension culture. HeLa cells were grown as suspension cells cultured in shaker flasks on top of rotary shakers set at 80-100 rpm. Cells were seeded at 1-4×10⁵ cells/l. The cells were allowed to grow to a cell concentration of 1-3×10⁶ cells/ml before splitting down to 1-4×10⁵ cells/ml. Suspension cells in the healthy growth phase (mid-log) were used for protein production use. In certain embodiments, the media may be supplemented with heparin to prevent aggregation of cells. This cell culture system allows for some increase of cell density while cell viability is maintained. Once the cells are growing in culture, they are passaged approximately 7 times in the spinner flasks.

E. Cell Culture Systems

The present invention will take advantage of the recently available bioreactor technology. Growing cells according to the present invention in a bioreactor allows for large scale production of fully biologically-active cells capable of being infected by the adenoviral vectors of the present invention.

As used herein, a “bioreactor” refers to any apparatus that can be used for the purpose of culturing cells. Growing cells according to the present invention in a bioreactor allows for large scale production of fully biologically active cells capable of being infected by the adenoviral vectors of the present invention.

Bioreactors have been widely used for the production of biological products from both suspension and anchorage dependent animal cell cultures. For example, the most widely used producer cells for adenoviral vector production are anchorage dependent human embryonic kidney cells (293 cells). Microcarrier cell culture in stirred tank bioreactor provides very high volume-specific culture surface area and has been used for the production of viral vaccines (Griffiths, 1986). Furthermore, stirred tank bioreactors have industrially proven to be scaleable. The multiplate CellCube™ cell culture system manufactured by Corning-Costar also offers a very high volume specific surface area. Cells grow on both sides of the culture plates hermetically sealed together in the shape of a compact cube. Unlike stirred tank bioreactors, the CellCube™ culture unit is disposable.

Another example of a bioreactor that may be employed in the present invention is a Wave Bioreactor®. The Wave Bioreactor® can be a Wave Biotech® model20/50EH. According to a particular aspect of the invention, the Wave Bioreactor® is used with serum-free media. As used herein, “media” and “medium” refers to any substance which can facilitate growth of host cells. According to one aspect of the present invention, the host cells are grown in media that is serum-free media. One example of a protein-free media is CD293. Another example of media that can support host cell growth is DMEM+2% FBS. One of skill in the art would understand that various components and agents can be added to the media to facilitate and control cell growth.

1. Anchorage-Dependent Versus Non-Anchorage-Dependent Cultures

Animal and human cells can be propagated in vitro in two modes: as non-anchorage dependent cells growing freely in suspension throughout the bulk of the culture; or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. Large scale suspension culture based on microbial (bacterial and yeast) fermentation technology has clear advantages for the manufacturing of mammalian cell products. The processes are relatively simple to operate and straightforward to scale up. Homogeneous conditions can be provided in the reactor which allows for precise monitoring and control of temperature, dissolved oxygen, and pH, and ensures that representative samples of the culture can be taken.

2. Reactors and Processes for Suspension

Large scale suspension culture of mammalian cells in stirred tanks was undertaken. The instrumentation and controls for bioreactors adapted, along with the design of the fermentors, from related microbial applications. However, acknowledging the increased demand for contamination control in the slower growing mammalian cultures, improved aseptic designs were quickly implemented, improving dependability of these reactors. Instrumentation and controls are basically the same as found in other fermentors and include agitation, temperature, dissolved oxygen, and pH controls. More advanced probes and autoanalyzers for on-line and off-line measurements of turbidity (a function of particles present), capacitance (a function of viable cells present), glucose/lactate, carbonate/bicarbonate and carbon dioxide are available. Maximum cell densities obtainable in suspension cultures are relatively low at about 2-4×10⁶ cells/ml of medium (which is less than 1 mg dry cell weight per ml), well below the numbers achieved in microbial fermentation.

Two suspension culture reactor designs are most widely used in the industry due to their simplicity and robustness of operation—the stirred reactor and the airlift reactor. The stirred reactor design has successfully been used on a scale of 8000 liter capacity for the production of interferon (Phillips et al., 1985; Mizrahi, 1983). Cells are grown in a stainless steel tank with a height-to-diameter ratio of 1:1 to 3:1. The culture is usually mixed with one or more agitators, based on bladed disks or marine propeller patterns. Agitator systems offering less shear forces than blades have been described. Agitation may be driven either directly or indirectly by magnetically coupled drives. Indirect drives reduce the risk of microbial contamination through seals on stirrer shafts.

The airlift reactor, also initially described for microbial fermentation and later adapted for mammalian culture, relies on a gas stream to both mix and oxygenate the culture. The gas stream enters a riser section of the reactor and drives circulation. Gas disengages at the culture surface, causing denser liquid free of gas bubbles to travel downward in the downcomer section of the reactor. The main advantage of this design is the simplicity and lack of need for mechanical mixing. Typically, the height-to-diameter ratio is 10:1. The airlift reactor scales up relatively easily, has good mass transfer of gasses and generates relatively low shear forces.

Most large-scale suspension cultures are operated as batch or fed-batch processes because they are the most straightforward to operate and scale up. However, continuous processes based on chemostat or perfusion principles are available.

A batch process is a closed system in which a typical growth profile is seen. A lag phase is followed by exponential, stationary and decline phases. In such a system, the environment is continuously changing as nutrients are depleted and metabolites accumulate. This makes analysis of factors influencing cell growth and productivity, and hence optimization of the process, a complex task. Productivity of a batch process may be increased by controlled feeding of key nutrients to prolong the growth cycle. Such a fed-batch process is still a closed system because cells, products and waste products are not removed.

In what is still a closed system, perfusion of fresh medium through the culture can be achieved by retaining the cells with a variety of devices (e.g. fine mesh spin filter, hollow fiber or flat plate membrane filters, settling tubes). Spin filter cultures can produce cell densities of approximately 5×10⁷ cells/ml. A true open system and the simplest perfusion process is the chemostat in which there is an inflow of medium and an outflow of cells and products. Culture medium is fed to the reactor at a predetermined and constant rate which maintains the dilution rate of the culture at a value less than the maximum specific growth rate of the cells (to prevent washout of the cell mass from the reactor). Culture fluid containing cells and cell products and byproducts is removed at the same rate.

3. Non-Perfused Attachment Systems

Traditionally, anchorage-dependent cell cultures are propagated on the bottom of small glass or plastic vessels. The restricted surface-to-volume ratio offered by classical and traditional techniques, suitable for the laboratory scale, has created a bottleneck in the production of cells and cell products on a large scale. In an attempt to provide systems that offer large accessible surfaces for cell growth in small culture volume, a number of techniques have been proposed: the roller bottle system, the stack plates propagator, the spiral film bottles, the hollow fiber system, the packed bed, the plate exchanger system, and the membrane tubing reel. Since these systems are non-homogeneous in their nature, and are sometimes based on multiple processes, they suffer from the following shortcomings—limited potential for scale-up, difficulties in taking cell samples, limited potential for measuring and controlling key process parameters and difficulty in maintaining homogeneous environmental conditions throughout the culture.

Despite these drawbacks, a commonly used process for large scale anchorage-dependent cell production is the roller bottle. Being little more than a large, differently shaped T-flask, simplicity of the system makes it very dependable and, hence, attractive. Fully automated robots are available that can handle thousands of roller bottles per day, thus eliminating the risk of contamination and inconsistency associated with the otherwise required intense human handling. With frequent media changes, roller bottle cultures can achieve cell densities of close to 0.5×10⁶ cells/cm² (corresponding to approximately 10⁹ cells/bottle or almost 10⁷ cells/ml of culture media).

4. Cultures on Microcarriers

In an effort to overcome the shortcomings of the traditional anchorage-dependent culture processes, van Wezel (1967) developed the concept of the microcarrier culturing systems. In this system, cells are propagated on the surface of small solid particles suspended in the growth medium by slow agitation. Cells attach to the microcarriers and grow gradually to confluency on the microcarrier surface. In fact, this large scale culture system upgrades the attachment dependent culture from a single disc process to a unit process in which both monolayer and suspension culture have been brought together. Thus, combining the necessary surface for a cell to grow with the advantages of the homogeneous suspension culture increases production.

The advantages of microcarrier cultures over most other anchorage-dependent, large-scale cultivation methods are several fold. First, microcarrier cultures offer a high surface-to-volume ratio (variable by changing the carrier concentration) which leads to high cell density yields and a potential for obtaining highly concentrated cell products. Cell yields are up to 1-2×10⁷ cells/ml when cultures are propagated in a perfused reactor mode. Second, cells can be propagated in one unit process vessels instead of using many small low-productivity vessels (i.e., flasks or dishes). This results in far better nutrient utilization and a considerable saving of culture medium. Moreover, propagation in a single reactor leads to reduction in need for facility space and in the number of handling steps required per cell, thus reducing labor cost and risk of contamination. Third, the well-mixed and homogeneous microcarrier suspension culture makes it possible to monitor and control environmental conditions (e.g., pH, pO2, and concentration of medium components), thus leading to more reproducible cell propagation and product recovery. Fourth, it is possible to take a representative sample for microscopic observation, chemical testing, or enumeration. Fifth, since microcarriers settle out of suspension quickly, use of a fed-batch process or harvesting of cells can be done relatively easily. Sixth, the mode of the anchorage-dependent culture propagation on the microcarriers makes it possible to use this system for other cellular manipulations, such as cell transfer without the use of proteolytic enzymes, cocultivation of cells, transplantation into animals, and perfusion of the culture using decanters, columns, fluidized beds, or hollow fibers for microcarrier retainment. Seventh, microcarrier cultures are relatively easily scaled up using conventional equipment used for cultivation of microbial and animal cells in suspension.

5. Microencapsulation of Mammalian Cells

One method which has shown to be particularly useful for culturing mammalian cells is microencapsulation. The mammalian cells are retained inside a semipermeable hydrogel membrane. A porous membrane is formed around the cells permitting the exchange of nutrients, gases, and metabolic products with the bulk medium surrounding the capsule. Several methods have been developed that are gentle, rapid and non-toxic and where the resulting membrane is sufficiently porous and strong to sustain the growing cell mass throughout the term of the culture. These methods are all based on soluble alginate gelled by droplet contact with a calcium-containing solution. U.S. Pat. No. 4,352,883, incorporated herein by reference, describes cells concentrated in an approximately 1% solution of sodium alginate which are forced through a small orifice, forming droplets, and breaking free into an approximately 1% calcium chloride solution. The droplets are then cast in a layer of polyamino acid that ionically bonds to the surface alginate. Finally the alginate is reliquefied by treating the droplet in a chelating agent to remove the calcium ions. Other methods use cells in a calcium solution to be dropped into a alginate solution, thus creating a hollow alginate sphere. A similar approach involves cells in a chitosan solution dropped into alginate, also creating hollow spheres.

Microencapsulated cells are easily propagated in stirred tank reactors and, with beads sizes in the range of 150-1500 μm in diameter, are easily retained in a perfused reactor using a fine-meshed screen. The ratio of capsule volume to total media volume can be maintained from as dense as 1:2 to 1:10. With intracapsular cell densities of up to 108, the effective cell density in the culture is 1-5×10⁷.

The advantages of microencapsulation over other processes include the protection from the deleterious effects of shear stresses which occur from sparging and agitation, the ability to easily retain beads for the purpose of using perfused systems, scale up is relatively straightforward and the ability to use the beads for implantation.

The current invention includes cells which are anchorage-dependent in nature. HeLa cells, for example, are anchorage-dependent, and when grown in suspension, the cells will attach to each other and grow in clumps, eventually suffocating cells in the inner core of each clump as they reach a size that leaves the core cells unsustainable by the culture conditions.

### IV. ADENOVIRAL VECTORS

The methods of the present invention involve expression constructs of the therapeutic nucleic acids comprised in adenoviral vectors for delivery of the nucleic acid. Although adenovirus vectors are known to have a low capacity for integration into genomic DNA, this feature is counterbalanced by the high efficiency of gene transfer afforded by these vectors. The vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.), is particularly efficient during the late phase of infection, and all the mRNA's issued from this promoter possess a 5′-tripartite leader (TPL) sequence which makes them useful mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

A second aspect of the present invention is the generation of replication-defective adenoviral vectors comprising a nucleic acid encoding a heterologous protein of interest. Accordingly, the vector possesses deletions adenoviral genome necessary for viral replication. Generally, a deletion encompasses the E1 region of the adenoviral genome. Since the E3 region is dispensable from the adenoviral genome, this portion may be deleted as well (Jones and Shenk, 1978). Therefore, current replication-defective adenoviral vectors carry heterologous DNA in either the deleted E1 region, the E3 region, or both regions. However, these adenoviral vectors are designed such that replication is possible when combined with a helper cells, such as the 293 cell line, which was transformed from human embryonic kidney cells by Ad5 DNA fragments and constitutively express the E1 proteins (Graham et al., 1977). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

The adenoviral vector may be of any of the 42 different known serotypes or subgroups A-F. Adenovirus type 5 of subgroup C is a particular starting material in order to obtain the replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication-defective and will not have an adenovirus E1 region. Thus, it will be most convenient to introduce the heterologous gene at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

The present invention employs, in one example, replication-defective adenoviral vector infection of cells in order to generate heterologous protein encoded by the vector. Typically, the adenoviral vector will simply be exposed to the appropriate host cell under physiologic conditions, permitting uptake of the virus. In certain embodiments of the present invention, the methods for producing a heterologous protein will involve initiating virus infection by diluting the host cells with fresh media and adenovirus. This avoids the need for a separate medium exchange step prior to infection. The invention contemplates that any amount of dilution of the host cells is contemplated by the present invention. In certain embodiments, the host cells are diluted 10-fold with fresh media. The invention also contemplates any amount of virus added to initiate infection. For example, virus infection may be initiated by adding 500 vp/host cell. The embodiments of the present invention also contemplate that virus infection can be allowed to proceed for any length of time.

### VI. METHODS OF HETEROLOGOUS PROTEIN HARVEST

Normally, adenoviral infection results in the lysis of cells being infected. However, when non trans-complementing cells are infected with replication-defective adenoviral vectors, no adenoviral particles are produced and this method may not be relied upon. Therefore, in order to harvest heterologous protein produced by adenoviral vectors, two different methods may be employed. If the protein(s) of interest encoded by the adenoviral vector comprising a heterologous gene is secreted by the infected cells, the cellular supernatant may be harvested directly. Alternatively, if the protein(s) of interest encoded by the adenoviral vector comprising a heterologous gene is not secreted, the cells may be harvested and lysed to extract the desired protein(s). Table 4 lists the most common methods that have been used for lysing cells after cell harvest.

TABLE 4

| Methods | Procedures | Comments |
|---|---|---|
| Freeze-thaw | Cycling between | Easy to carry out at lab |
| | dry ice and | scale. High cell |
| | 37° C. water bath | lysis efficiency |
| | | Not scaleable |
| | | Not recommended for large |
| | | scale manufacturing |
| Solid Shear | French Press | Capital equipment |
| | Hughes Press | investment |
| | | Virus containment concerns |
| | | Lack of experience |
| Detergent | Non-ionic detergent | Easy to carry out at both lab |
| lysis | solutions such as | and manufacturing |
| | Tween, Triton, NP-40, | scale |
| | etc. | Wide variety of detergent |
| | | choices |
| | | Concerns of residual |
| | | detergent in finished |
| | | product |
| Hypotonic | water, citric buffer | Low lysis efficiency |
| solution | | |
| lysis | | |
| Liquid Shear | Homogenizer | Capital equipment |
| | Impinging Jet | investment |
| | Microfluidizer | Virus containment concerns |
| | | Scaleability concerns |
| Sonication | Ultrasound | Capital equipment |
| | | investment |
| | | Virus containment concerns |
| | | Noise pollution |
| | | Scaleability concern |

A. Detergents

Cells are bounded by membranes. In order to release components of the cell, it is necessary to break open the cells. The most advantageous way in which this can be accomplished, according to the present invention, is to solubilize the membranes with the use of detergents. Detergents are amphipathic molecules with an apolar end of aliphatic or aromatic nature and a polar end which may be charged or uncharged. Detergents are more hydrophilic than lipids and thus have greater water solubility than lipids. They allow for the dispersion of water insoluble compounds into aqueous media and are used to isolate and purify proteins in a native form.

Detergents can be denaturing or non-denaturing. The former can be anionic such as sodium dodecyl sulfate or cationic such as ethyl trimethyl ammonium bromide. These detergents totally disrupt membranes and denature the protein by breaking protein-protein interactions. Non denaturing detergents can be divided into non-anionic detergents such as Triton®X-100, bile salts such as cholates and zwitterionic detergents such as CHAPS. Zwitterionics contain both cationic and anion groups in the same molecule, the positive electric charge is neutralized by the negative charge on the same or adjacent molecule.

Denaturing agents such as SDS bind to proteins as monomers and the reaction is equilibrium driven until saturated. Thus, the free concentration of monomers determines the necessary detergent concentration. SDS binding is cooperative i.e. the binding of one molecule of SDS increase the probability of another molecule binding to that protein, and alters proteins into rods whose length is proportional to their molecular weight.

Non-denaturing agents such as Triton®X-100 do not bind to native conformations nor do they have a cooperative binding mechanism. These detergents have rigid and bulky apolar moieties that do not penetrate into water soluble proteins. They bind to the hydrophobic parts of proteins. Triton®X-100 and other polyoxyethylene nonanionic detergents are inefficient in breaking protein-protein interaction and can cause artifactual aggregations of protein. These detergents will, however, disrupt protein-lipid interactions but are much gentler and capable of maintaining the native form and functional capabilities of the proteins.

Detergent removal can be attempted in a number of ways. Dialysis works well with detergents that exist as monomers. Dialysis is somewhat ineffective with detergents that readily aggregate to form micelles because the micelles are too large to pass through dialysis. Ion exchange chromatography can be utilized to circumvent this problem. The disrupted protein solution is applied to an ion exchange chromatography column and the column is then washed with buffer minus detergent. The detergent will be removed as a result of the equilibration of the buffer with the detergent solution. Alternatively the protein solution may be passed through a density gradient. As the protein sediments through the gradients the detergent will come off due to the chemical potential.

Often a single detergent is not versatile enough for the solubilization and analysis of the milieu of proteins found in a cell. The proteins can be solubilized in one detergent and then placed in another suitable detergent for protein analysis. The protein detergent micelles formed in the first step should separate from pure detergent micelles. When these are added to an excess of the detergent for analysis, the protein is found in micelles with both detergents. Separation of the detergent-protein micelles can be accomplished with ion exchange or gel filtration chromatography, dialysis or buoyant density type separations.

Triton®X-Detergents: This family of detergents (Triton®X-100, X114 and NP-40) have the same basic characteristics but are different in their specific hydrophobic-hydrophilic nature. All of these heterogeneous detergents have a branched 8-carbon chain attached to an aromatic ring. This portion of the molecule contributes most of the hydrophobic nature of the detergent. Triton®X detergents are used to solubilize membrane proteins under non-denaturing conditions. The choice of detergent to solubilize proteins will depend on the hydrophobic nature of the protein to be solubilized. Hydrophobic proteins require hydrophobic detergents to effectively solubilize them.

Triton®X-100 and NP-40 are very similar in structure and hydrophobicity and are interchangeable in most applications including cell lysis, delipidation protein dissociation and membrane protein and lipid solubilization. Generally 2 mg detergent is used to solubilize 1 mg membrane protein or 10 mg detergent/1 mg of lipid membrane. Triton®X-114 is useful for separating hydrophobic from hydrophilic proteins.

Brij® Detergents: These are similar in structure to Triton®X detergents in that they have varying lengths of polyoxyethylene chains attached to a hydrophobic chain. However, unlike Triton®X detergents, the Brij® detergents do not have an aromatic ring and the length of the carbon chains can vary. The Brij® detergents are difficult to remove from solution using dialysis but may be removed by detergent removing gels. Brij®58 is most similar to Triton X100 in its hydrophobic/hydrophilic characteristics. Brij®-35 is a commonly used detergent in HPLC applications.

Dializable Nonionic Detergents: η-Octyl-β-D-glucoside (octylglucopyranoside) and η-Octyl-β-D-thioglucoside (octylthioglucopyranoside, OTG) are nondenaturing nonionic detergents which are easily dialyzed from solution. These detergents are useful for solubilizing membrane proteins and have low UV absorbances at 280 nm. Octylglucoside has a high CMC of 23-25 mM and has been used at concentrations of 1.1-1.2% to solubilize membrane proteins.

Octylthioglucoside was first synthesized to offer an alternative to octylglucoside. Octylglucoside is expensive to manufacture and there are some inherent problems in biological systems because it can be hydrolyzed by β-glucosidase.

Tween® Detergents: The Tweene detergents are nondenaturing, nonionic detergents. They are polyoxyethylene sorbitan esters of fatty acids. Tween® 20 and Tween® 80 detergents are used as blocking agents in biochemical applications and are usually added to protein solutions to prevent nonspecific binding to hydrophobic materials such as plastics or nitrocellulose. They have been used as blocking agents in ELISA and blotting applications. Generally, these detergents are used at concentrations of 0.01-1.0% to prevent nonspecific binding to hydrophobic materials.

Tween® 20 and other nonionic detergents have been shown to remove some proteins from the surface of nitrocellulose. Tween® 80 has been used to solubilize membrane proteins, present nonspecific binding of protein to multiwell plastic tissue culture plates and to reduce nonspecific binding by serum proteins and biotinylated protein A to polystyrene plates in ELISA.

The difference between these detergents is the length of the fatty acid chain. Tween® 80 is derived from oleic acid with a C₁₈ chain while Tween® 20 is derived from lauric acid with a C₁₂ chain. The longer fatty acid chain makes the Tween® 80 detergent less hydrophilic than Tween® 20 detergent. Both detergents are very soluble in water.

The Tween® detergents are difficult to remove from solution by dialysis, but Tween® 20 can be removed by detergent removing gels. The polyoxyethylene chain found in these detergents makes them subject to oxidation (peroxide formation) as is true with the Triton® X and Brij® series detergents.

Zwitterionic Detergents: The zwitterionic detergent, CHAPS, is a sulfobetaine derivative of cholic acid. This zwitterionic detergent is useful for membrane protein solubilization when protein activity is important. This detergent is useful over a wide range of pH (pH 2-12) and is easily removed from solution by dialysis due to high CMCs (8-10 mM). This detergent has low absorbances at 280 nm making it useful when protein monitoring at this wavelength is necessary. CHAPS is compatible with the BCA Protein Assay and can be removed from solution by detergent removing gel. Proteins can be iodinated in the presence of CHAPS

CHAPS has been successfully used to solubilize intrinsic membrane proteins and receptors and maintain the functional capability of the protein. When cytochrome P-450 is solubilized in either Triton® X-100 or sodium cholate aggregates are formed.

B. Non-Detergent Methods

Various non-detergent methods, though not preferred, may be employed in conjunction with other advantageous aspects of the present invention:

Freeze-Thaw: This has been a widely used technique for lysis cells in a gentle and effective manner. Cells are generally frozen rapidly in, for example, a dry ice/ethanol bath until completely frozen, then transferred to a 37° C. bath until completely thawed. This cycle is repeated a number of times to achieve complete cell lysis.

Sonication: High frequency ultrasonic oscillations have been found to be useful for cell disruption. The method by which ultrasonic waves break cells is not fully understood but it is known that high transient pressures are produced when suspensions are subjected to ultrasonic vibration. The main disadvantage with this technique is that considerable amounts of heat are generated. In order to minimize heat effects specifically designed glass vessels are used to hold the cell suspension. Such designs allow the suspension to circulate away from the ultrasonic probe to the outside of the vessel where it is cooled as the flask is suspended in ice.

High Pressure Extrusion: This is a frequently used method to disrupt microbial cell. The French pressure cell employs pressures of 10.4×10⁷ Pa (16,000 p.s.i) to break cells open. These apparatus consists of a stainless steel chamber which opens to the outside by means of a needle valve. The cell suspension is placed in the chamber with the needle valve in the closed position. After inverting the chamber, the valve is opened and the piston pushed in to force out any air in the chamber. With the valve in the closed position, the chamber is restored to its original position, placed on a solid based and the required pressure is exerted on the piston by a hydraulic press. When the pressure has been attained the needle valve is opened fractionally to slightly release the pressure and as the cells expand they burst. The valve is kept open while the pressure is maintained so that there is a trickle of ruptured cell which may be collected.

Solid Shear Methods: Mechanical shearing with abrasives may be achieved in Mickle shakers which oscillate suspension vigorously (300-3000 time/min) in the presence of glass beads of 500 nm diameter. This method may result in organelle damage. A more controlled method is to use a Hughes press where a piston forces most cells together with abrasives or deep frozen paste of cells through a 0.25 mm diameter slot in the pressure chamber. Pressures of up to 5.5×10⁷ Pa (8000 p.s.i.) may be used to lyse bacterial preparations.

Liquid Shear Methods: These methods employ blenders, which use high speed reciprocating or rotating blades, homogenizers which use an upward/downward motion of a plunger and ball and microfluidizers or impinging jets which use high velocity passage through small diameter tubes or high velocity impingement of two fluid streams. The blades of blenders are inclined at different angles to permit efficient mixing. Homogenizers are usually operated in short high speed bursts of a few seconds to minimize local heat. These techniques are not generally suitable for microbial cells but even very gentle liquid shear is usually adequate to disrupt animal cells.

Hypotonic/Hypertonic Methods: Cells are exposed to a solution with a much lower (hypotonic) or higher (hypertonic) solute concentration. The difference in solute concentration creates an osmotic pressure gradient. The resulting flow of water into the cell in a hypotonic environment causes the cells to swell and burst. The flow of water out of the cell in a hypertonic environment causes the cells to shrink and subsequently burst.

### VII. METHODS OF CONCENTRATION AND FILTRATION

The present invention involves methods of producing heterologous proteins derived from heterologous nucleic acid expression constructs encoded by adenoviral vectors. Methods of isolating heterologous proteins from host cells include any methods known to those of skill in the art. For example, these methods may include clarification, concentration and diafiltration. One step in the purification process can include clarification of the cell lysate to remove large particulate matter, particularly cellular components, from the cell lysate. Clarification of the lysate can be achieved using a depth filter or by tangential flow filtration. In one embodiment of the present invention, the cell lysate is concentrated. Concentrating the crude cell lysate may include any step known to those of skill in the art. For example, the crude cell lysate may be passed through a depth filter, which consists of a packed column of relatively non-adsorbent material (e.g., polyester resins, sand, diatomaceous earth, colloids, gels and the like). In tangential flow filtration (TFF), the lysate solution flows across a membrane surface which facilitates back diffusion of solute from the membrane surface into the bulk solution. Membranes are generally arranged within various types of filter apparatus including open channel plate and frame, hollow fibers, and tubules.

After clarification and prefiltration of the cell lysate, the resultant heterologous protein supernatant may be concentrated and buffer may be exchanged by diafiltration. The protein supernatant can be concentrated by tangential flow filtration across an ultrafiltration membrane of 10-30K nominal molecular weight cutoff. Ultrafiltration is a pressure modified convective process that uses semi-permeable membranes to separate species by molecular size, shape and/or charge. It separates solvents from solutes of various sizes independent of solute molecular size. Ultrafiltration is gentle, efficient and can be used to simultaneously concentrate and desalt solutions. Ultrafiltration membranes generally have two distinct layers: a thin, dense skin and an open structure of progressively larger voids which are largely open to the permeate side of the ultrafilter. Any species capable of passing through the pores of the skin can therefore freely pass through the membrane. For maximum retention of solute, a membrane is selected that has a nominal molecular weight cut-off well below that of the species being retained. In macromolecular concentration, the membrane enriches the content of the desired biological species and provides filtrate cleared of retained substances. Microsolutes are removed convectively with the solvent. As concentration of the retained solute increases, the ultrafiltration rate diminishes.

In some embodiments of the present invention, an exchange buffer may be used. Buffer exchange, or diafiltration, involving ultrafilters, may be used for the removal and exchange of salts, sugars, non-aqueous solvents or material of low molecular weight.

### VIII. METHODS OF PROTEIN PURIFICATION

It may be desirable to purify the heterologous protein(s) produced by the adenoviral vector comprising a heterologous gene. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; sodium dodecyl sulfate/polyacrylamide gel electrophoresis (SDS/PAGE); isoelectric focusing. A particularly efficient method of purifying peptides is fast protein liquid chromatography (FPLC) or even HPLC.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample can be low because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding. It should be possible to elute the substance without destroying the sample or the ligand. One of the most common forms of affinity chromatography is immunoaffinity chromatography. The generation of antibodies that would be suitable for use in accord with the present invention is discussed below.

The use of antibodies of the present invention, in an ELISA assay is contemplated. For example, antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for the protein of interest that differs the first antibody. Appropriate conditions preferably include diluting the sample with diluents such as BSA, bovine gamma globulin (BGG) and phosphate buffered saline (PBS)/Tween®. These added agents also tend to assist in the reduction of nonspecific background. The layered antisera is then allowed to incubate for from about 2 to about 4 hr, at temperatures preferably on the order of about 25° C. to about 27° C. Following incubation, the antisera-contacted surface is washed so as to remove non-immunocomplexed material. A particular procedure includes washing with a solution such as PBS/Tween®, or borate buffer.

Hydrophobic Interaction Chromatography (HIC) is based on hydrophobic attraction between the stationary phase and the protein molecules. The stationary phase consists of small non-polar groups (butyl, octyl or phenyl) attached to a hydrophilic polymer backbone (cross-linked dextran or agarose, for example). Separations by HIC are often designed using nearly opposite conditions to those used in ion exchange chromatography. Binding of the proteins is often carried out at high salt concentration. Some proteins may precipitate at this high ionic strength, thus necessitating removal by centrifugation prior to loading the protein mixture onto the column. Selective elution of bound proteins is then carried out by applying a decreasing salt gradient.

Any suitable chromatographic material can be used. For example, a variety of different chromatographic materials supports are commercially available which have hydrophobic ligands attached to a chromatographic support. For example, the ligands may have an alkyl chain ranging from about two to twenty or more carbons in length. These ligands may be branched, linear, or contain carbon rings, such as phenyl rings. Increasing chain length typically results in a chromatographic medium with greather hydrophobicity. Commonly used ligands are phenyl-, butyl-, and octyl-residues. Commercially available hydrophobic interaction chromatographic materials include, but are not limited to: POROS HP2′, POROS PE“and POROS ET” (Applied Biosystems, Foster City, Calif.); Bio-Rad Macro-Prep HIC Supports, Bio-Rad Methyl HIC support, Bio-Rad-t-butyl HIC support, Bio-rad Econo column butyl-650m (Bio-Rad, Hercules, Calif.) TosoHaas TSK-GELO and TosoHaas TOKYOPEARL (Tosh Bioscience, Montgomeryville, Pa.); Fractogel EMD Propyl (S) and EMD Phenyl I (S) (Merck, Darmstadt, Germany); IEC PH-814 (Phenomenex, Torrence, Calif.) and HiPrep 16/10 Phenyl, HiPrep™ 16/1-Butyl, HiPrep™ 16/10 Octyl, HiLoad Phenyl-Sepharose FF and HiLoad Butyl-Sepharose FF (GE Helthcare, Little Chalfont, UK).

A further detailed description of the general principles of hydrophobic interaction chromatography media may be found in U.S. Pat. No. 3,917,527 and in U.S. Pat. No. 4,000,098. Other examples of HIC purification of specific proteins may be found, for example, in the following references: U.S. Pat. No. 4,332,717 (human growth hormone), U.S. Pat. No. 4,771,128 (toxin conjugates), U.S. Pat. No. 4,743,680 (antihemolytic factor), U.S. Pat. No. 4,894,439 (tumor necrosis factor), U.S. Pat. No. 4,908,434 (11-2), U.S. Pat. No. 4,920,196 (human lymphotoxin) and Fausnaugh and Regnier, 1986 (lysozyme species) and U.S. Pat. No. 5,252,216 (soluable complement receptors), each of which is herein incorporated by reference.

Hydroxyapatite chromatography is a method of purifying proteins that utilizes an insoluble hydroxylated calcium phosphate which forms both the matrix and ligand. Functional groups consist of pairs of positively charged calcium ions (C-sites) and clusters of negatively charged phosphate groups (P-sites).

Various hydroxyapatite chromatographic resins are available commercially, and any available form of the material can be used in the practice of this invention. In one embodiment of the invention, the hydroxyapatite is in a crystalline form. Hydroxyapatites for use in this invention may be those that are agglomerated to form particles and sintered at high temperatures into a stable porous ceramic mass.

A number of chromatographic supports may be employed in the preparation of hydroxyapatite chromatography columns, the most extensively used are Type I and Type II hydroxyapatite. Type I has a high protein binding capacity and better capacity for acidic proteins. Type II, however, has a lower protein binding capacity, but has better resolution of nucleic acids and certain proteins. The Type II material also has a very low affinity for albumin and is especially suitable for the purification of many species and classes of immunoglobulins. The choice of an application appropriate hydroxyapatite may be determined by those of skill in the art. Commercially available hydroxyapatite chromatographic materials include, but are not limited to: CHT™ Ceramic Hydroxyapatite, Type I, (20, 40 or 80 μm) and CHT™ Ceramic Hydroxyapatite, Type II, (20, 40 or 80 μm) (Bio-Rad, Hercules, Calif.) and HA-Ultrogel® (Sigma-Aldrich, St. Louis, Mo.).

The present invention also may employ nucleases to remove contaminating nucleic acids. Exemplary nucleases include Benzonase®, Pulmozyme®; or any other DNase or RNase commonly used within the art.

Enzymes such as Benzonaze® degrade nucleic acid and have no proteolytic activity. The ability of Benzonase® to rapidly hydrolyze nucleic acids makes the enzyme ideal for reducing cell lysate viscosity. It is well known that nucleic acids may adhere to cell derived particles such as viruses. The adhesion may interfere with separation due to agglomeration, change in size of the particle or change in particle charge, resulting in little if any product being recovered with a given purification scheme. Benzonase® is well suited for reducing the nucleic acid load during purification, thus eliminating the interference and improving yield.

As with all endonucleases, Benzonase® hydrolyzes internal phosphodiester bonds between specific nucleotides. Upon complete digestion, all free nucleic acids present in solution are reduced to oligonucleotides 2 to 4 bases in length.

### IX. EXAMPLES

The following examples are included to demonstrate particular embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1

### Protein Production as a Function of Time

### A. Materials and Methods

1. Cell Lines And Culture

HeLa cells were obtained from American Type Culture Collection (ATCC, Rockville, Md.) and were adapted to grow in the CD293, serum free media. The suspension HeLa cells were grown as suspension cells in shaker flasks on top of rotary shakers set at 80-100 rpm inside an incubator at 37 C, 5-10% CO₂ and 90% humidity. Cells were seeded at 1-4×10⁵ cells/mL. The cells were allowed to grow to a cell concentration of 1-3×10⁶ cells/mL before splitting down to 1-4×10⁵ cells/mL. Suspension cells in the healthy growth phase (mid-log) were used for protein production use.

2. Recombinant Adenovirus

The recombinant adenovirus vectors carrying the mda-7 gene (Ad-mda7) was obtained from Introgen Therapeutics (Introgen Therapeutics, Houston, Tex.). Production of the replication-deficient human type 5 Adenovirus (Ad5) containing the mda-7 gene (Ad-mda7) has been previously reported (Saeki et al., 2000; Mhashilkar, 2001; Pataer et al., 2002). Construction of Ad-mda-7 involved linking mda-7 cDNA to a CMV-IE promoter, followed by an SV40 polyadenylation [p(A)] sequence; this expression cassette was placed in the E1 region of Ad5.

3. Viral Infection

HeLa suspension cells in culture were infected with Ad-mda7 virus (INGN241 P/N10-00015, L/N2119901) at a cell concentration of 1.3×10⁶ cells/ml. Multiplicity of infection (MOI) was 500 viral particles (vp)/cell. Samples of the culture media were collected at different time points after virus infection for MDA-7 protein analysis using western blot.

4. Western Blot

Total protein was isolated from the harvested cells by adding cell lysis buffer (20 mM HEPES, pH 7.5; 10 mM KCl, 1 mM MgCl2, 1 mM EDTA, 1 mM DTT, 250 mM sucrose and 1× protease inhibitor). Proteins were separated by SDS polyacrylamide gel electrophoresis and immobilized on nitrocellulose membranes. Membranes were blocked with 5% nonfat dry milk and incubated overnight at 4° C. with anti MDA-7 antibody (Introgen Therapeutics). Membranes were then washed and incubated with horseradish peroxidase (HRP)-conjugated secondary antibodies for 1 hr at room temperature. Following incubation, the membranes were developed and protein signals detected using enhanced chemilluminescence (ECL) western blotting detection reagents (Amersham Biosciences, Buckinghamshire, UK).

### B. Results

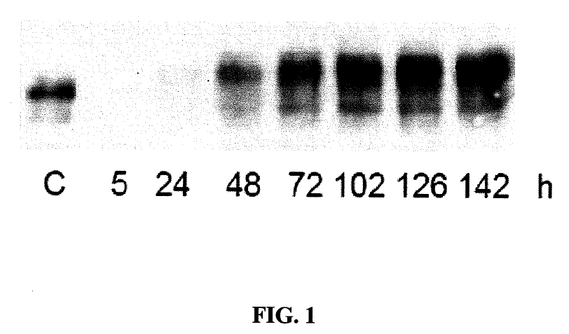

Levels of MDA-7 protein in the culture media increased as the virus infection proceeded. The highest levels of MDA-7 protein were observed after 72 hours of infection (FIG. 1). The results indicate that MDA-7 protein concentration in the infected HeLa culture media (post 72 hours infection) is significantly higher than the levels of mda7 protein expressed from a stably transfected 293 cells (named as 293M cells). Additionally, MDA-7 protein produced from HeLa cells appears to have a higher molecular weight than that from the stably transfected 293 cells. This is expected due to the different glycosylation patterns in the HeLa and 293 cells as the MDA-7 protein is known to be highly glycosylated. The MDA-7 protein isolated from the HeLa cell supernatant was found to be biologically active in inducing tumor cell death in culture.

A sample of the media harvest was also analyzed by HPLC to determine the level of adenovirus in the sample. No adenovirus was detected on the HPLC (limit of detection is 1E10 vp/mL (data not shown), suggesting no further adenovirus amplification during infection.

The data suggests that MDA-7 protein can be produced efficiently by infecting HeLa suspension cells with Ad-mda-7 virus. Concentration of MDA-7 protein is significantly higher than that achieved from a stably transfected 293 cells cells.

### Example 2

### Production of MDA-7 Protein in a Wave-20 Bioreactor

### A. Materials and Methods

1. Cell Culture Methodology

The HeLa suspension cells were maintained in the serum-free CD293 media inside an incubator at 37° C., 5-10% CO₂ and 90% humidity. The cells were seeded into a Wave-20 Bioreactor Wave Bioreactor (Wave Biotech, LLC, Bedminster, N.J.). Cells were allowed to grow inside the bioreactor under media perfusion to a cell concentration of 5.5×10⁷ cells/ml. The CD293 media was used for culture. At this point, the culture was diluted with fresh CD293 media to lower the cell concentration to 2.3×10⁶ cells/ml. Ten liters of the diluted culture was infected with Ad-mda-7 virus (P/N10-00030, L/P241001) at a MOI of 1000 vp/cell.

2. Recombinant Adenovirus

The recombinant adenovirus vectors carrying the mda-7 gene (Ad-mda7) was obtained from Introgen Therapeutics (Introgen Therapeutics, Houston, Tex.). Production of the replication-deficient human type 5 Adenovirus (Ad5) containing the mda-7 gene (Ad-mda7) has been previously reported (Saeki et al., 2000; Mhashilkar, 2001; Pataer et al., 2002). Construction of Ad-mda-7 involved linking mda-7 cDNA to a CMV-IE promoter, followed by an SV40 polyadenylation [p(A)] sequence; this expression cassette was placed in the E1 region of Ad5.

3. Viral Infection

Ten liters of the diluted culture was infected with Ad-mda-7 virus (P/N10-00030, L/P241001) at a MOI of 1000 vp/cell. Four days post infection, the culture supernatant was harvested. For comparison, a 293 suspension cell culture was also infected with the Ad-mda-7 virus and the culture media was harvested on days 2, 3, 4, 5 and 6. MDA-7 protein in the media harvest was subject to either centrifugation or filtration and analyzed by western blot as described in Example 1.

4. Western Blot

Total protein was isolated from the harvested cells by adding cell lysis buffer (20 mM HEPES, pH 7.5; 10 mM KCl, 1 mM MgCl₂, 1 mM EDTA, 1 mM DTT, 250 mM sucrose and 1× protease inhibitor). Proteins were separated by SDS polyacrylamide gel electrophoresis and immobilized on nitrocellulose membranes. Membranes were blocked with 5% nonfat dry milk and incubated overnight at 4° C. with anti MDA-7 (Introgen Therapeutics). Membranes were then washed and incubated with horseradish peroxidase (HRP)-conjugated secondary antibodies for 1 hr at room temperature. Following incubation, the membranes were developed and protein signals detected using enhanced chemilluminescence (ECL) western blotting detection reagents (Amersham Biosciences, Buckinghamshire, UK).

### B. Results

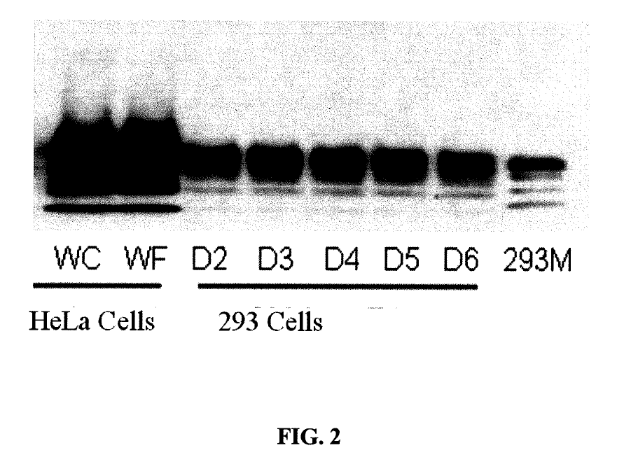

Very high levels of MDA-7 protein was found in both centrifuged and filtered portions of the HeLa cell media harvest as compared to MDA-7 protein found in Ad-mda-7 infected 293 cells (FIG. 2). Mda7 protein produced from the stably transfected 293M cells was used as a control.

### Example 3

### Production and Purification of MDA-7 Protein

### A. Materials and Methods

1. Cell Lines and Culture

HeLa cells were cultured in a Wave-20 Bioreactor using the methods described in Example 2.

2. Viral Infection and Cell Harvest

Ten liters of the diluted culture was infected with Ad-mda-7 (P/N 10-00015, L/N B2119901) at a MOI of 500 vp/cell. Four days post infection, the culture media was harvested. The harvest was clarified using a combination of one 10 inch 5.0 μm Polygard CN (P/N 01-00393, C/N 003648) and one 10 inch Polysep II 0.5 μm filters (P/N 01-00392, C/N 003773). The clarified harvest was concentrated 10-fold using a Pellicon 2 cassette with Biomax 100 kd membrane. After concentration, the material was immediately diafiltered against 5 volumes of DPBS. The concentrated and diafiltered mda-7 protein harvest was stored at <−60° C. for further purification study.

3. Protein Purification

Three chromatography protein purification techniques were evaluated using the previously collected MDA-7 protein. These chromatography protein purification techniques included 1) Phenyl-Sepharose FF hydrophobic interaction chromatography (HIC); 2) butyl-sepharose FF HIC; and 3) hydroxyapatite type 1 chromatography.

a. Phenyl-Sepharose FF Hydrophobic Interaction Chromatography

Phenyl-Sepharose FF (Amersham Pharmacia Cat# 17-0965-10, Lot# 277173) was packed inside a HR-16 column to a column volume of approximately 10 mL. The column was connected to an Akta explorer chromatography system (Amersham Pharmacia, model Akta explorer 100). Four buffers were used. Buffer A consisted of 20 mM phosphate+1M (NH₄)₂SO₄ pH 7.0. Buffer B consisted of 20 mM phosphate, pH 7.0. The Sanitization and regeneration buffer consisted of 1.0N NaOH. The storage solution consisted of 0.01N NaOH.

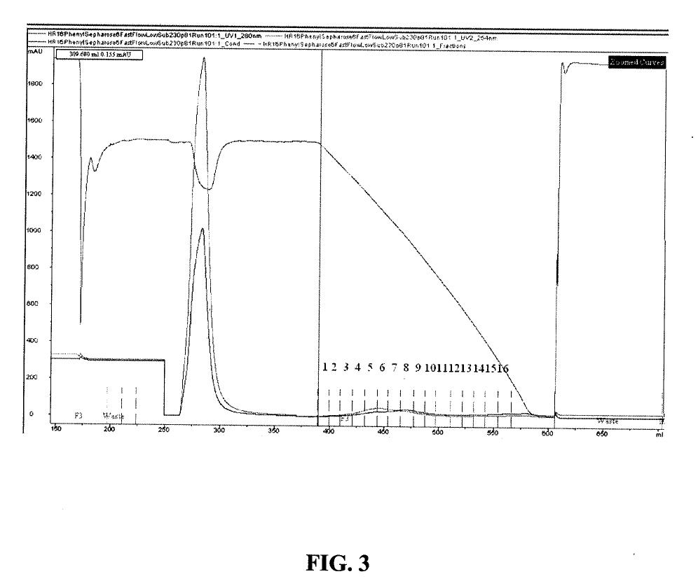

Briefly, the column was first sanitized with 1.0N NaOH solution for 35 minutes. After sanitization, the column was conditioned and equilibrated with 4 cv of buffer A. 15 mL of the stored MDA-7 material was thawed inside a 37° C. water bath. 5 mL of 3M ammonia sulfate solution was added to the material to a final (NH₄)₂SO₄ concentration of 1M. The material was loaded onto the equilibrated Phenyl-Sepharose FF column at 3 mL/min. At the completion of the loading, the column was washed with 5 cv of Buffer A to bring the UV absorbance to baseline. The column was eluted with a 15cv linear gradient from Buffer A to B. At the end of the elution, the column was regenerated using the sanitization and regeneration buffer and stored in 0.01N NaOH solution. During the linear elution step, 10 mL fractions were collected manually (FIG. 3). The collected fractions were analyzed by SDS-PAGE and MDA-7 Western blot as described in Example 1 to identify the fractions containing the MDA-7 protein.

b. Butyl-Sepharose FF Hydrophobic Interaction Chromatography

An alternative HIC resin Butyl-Sepharose FF resin (Amersham Pharmacia Cat# 170980-10, Lot# 306798) was also evaluated for the purification of MDA-7 protein. The resin was packed inside a XK-16 column to a column volume of approximately 10 mL. The column was connected to the Akta explorer chromatography system. The buffers used for the purification were the same as those used for the Phenyl-Sepharose FF column listed above.

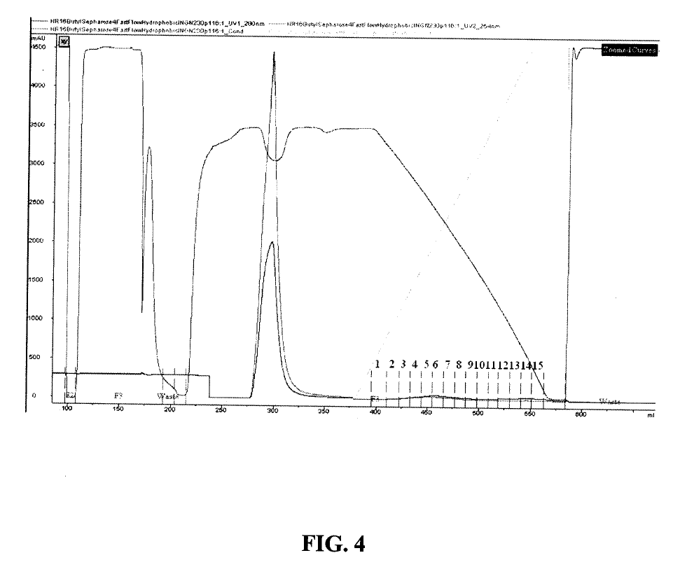

The column was first sanitized with 1.0N NaOH solution for 35 mins. After sanitization, the column was conditioned and equilibrated with 4 cv of Buffer A. 15 ml of stored mda-7 material was thawed inside a 37° C. water bath. 5 mL of 3M ammonia sulfate solution was added to the material to a final (NH₄)₂SO₄ concentration of 1M. The material was loaded onto the equilibrated Butyl-Sepharose FF column at 3 mL/min. At the completion of the loading, the column was washed with 5 cv of Buffer A to bring the UV absorbance to baseline. The column was eluted with a 15 cv linear gradient from Buffer A to B. During the linear elution step, 10 ml fractions were collected manually (FIG. 4). At the end of the elution, the column was regenerated using the Sanitization buffer and stored in 0.01N NaOH solution. The collected fractions were analyzed by SDS-PAGE and MDA-7 western blot as described in Example 1 to identify the fractions containing the MDA-7 protein.

c. Hydroxyapatite Type 1 Chromatography

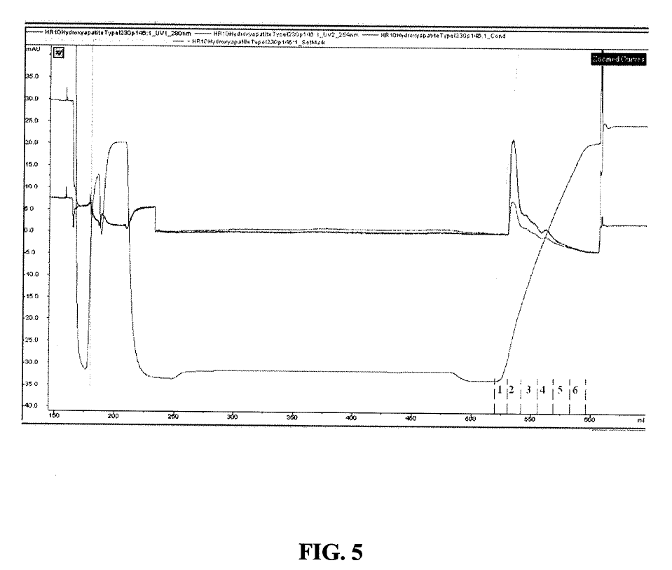

Due to the level of impurity in samples purified solely by butyl-sepharose FF hydrophobic interaction chromatography, it was decided to subject the eluate to a subsequent purification step consisting of hydroxyapatite type 1 chromatography. In accordance with this subsequent chromatography step, hydroxyapatite type 1 resin (Bio-Rad, Cat# 158-4000, Lot# 136182D) was packed inside a HR-16 column to a column volume of approximately 10 mL. The column was connected to the Akta explorer chromatography system. Four buffers were employed in this chromatography step. Buffer C consisted of 10 mM sodium phosphate, pH 6.8. Buffer D consisted of 0.45M sodium phosphate, pH 6.8. The sanitization and regeneration buffer and the storage solution were the same as described for the phenyl-sepharose FF hydrophobic interaction chromatography. Briefly, the column was first sanitized with 1.0N NaOH solution for 35 minutes. After sanitization, the column was equilibrated with 5 cv of buffer C. The MDA-7 protein fraction eluted from the Butyl-Sepharose FF column was diluted 5-fold with Buffer C. The diluted material was loaded onto the equilibrated hydroxyapatite type 1 column at 3 mL/min. At the completion of the loading, the column was washed with 5 cv of Buffer C to bring the UV absorbance to baseline. The column was eluted with a 10 cv linear gradient from Buffer C to D. At the end of the elution, the column was regenerated using the Sanitization buffer and stored in 0.01N NaOH solution. During the linear elution step, 10 mL fractions were collected manually (FIG. 5). At the end of the elution, the column was regenerated using the Sanitization buffer and stored in 0.01N NaOH solution. The collected fractions were analyzed by SDS-PAGE and MDA-7 western blot as described in Example 1 to identify the fractions containing the MDA-7 protein.

### B. Results

The results of the phenyl-sepharose FF hydrophobic interaction chromatography showed that the MDA-7 protein bound to the phenyl-sepharose FF column and was eluted out mostly in fractions 7, 8 and 9 between conductivity of 100 to 80 mS/cm. Significant amount of impurity proteins were removed by the phenyl-sepharose FF column, mostly in the flow through fraction.

Compared to the phenyl-sepharose FF column, MDA-7 protein had stronger interaction with the butyl-sepharose FF column. As a result, MDA-7 protein was eluted out towards the end of the elution gradient at a much lower conductivity. It appeared that the impurity protein bands in the MDA-7 protein containing fractions from the phenyl-sepharose FF and the butyl-sepharose FF columns were different. Furthermore, the salt concentration in the MDA-7 protein fraction from the phenyl-sepharose FF column was high enough to allow the MDA-7 protein to bind to the butyl-sepharose FF column without additional adjustment. Therefore, it was conceived to combine the 2 hydrophobic interaction chromatography columns in tandem to achieve additional mda-7 protein purification.

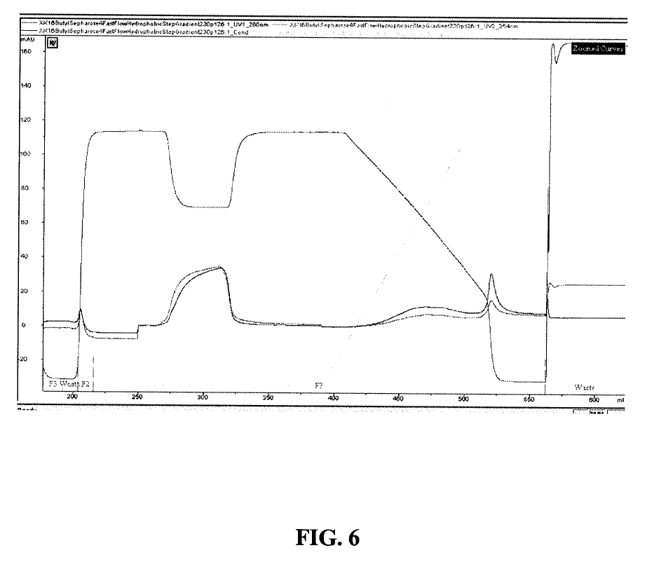

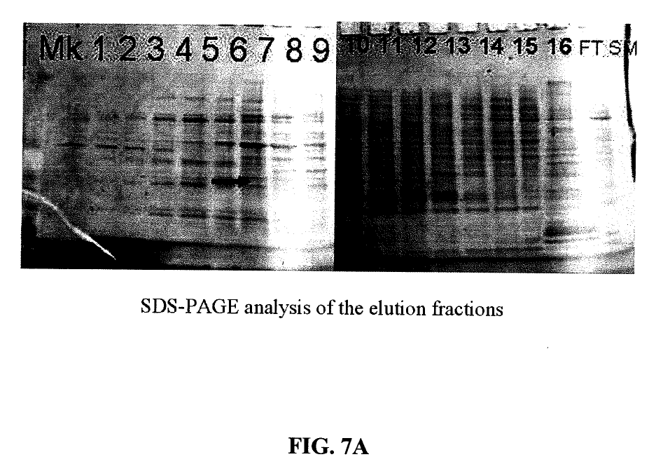

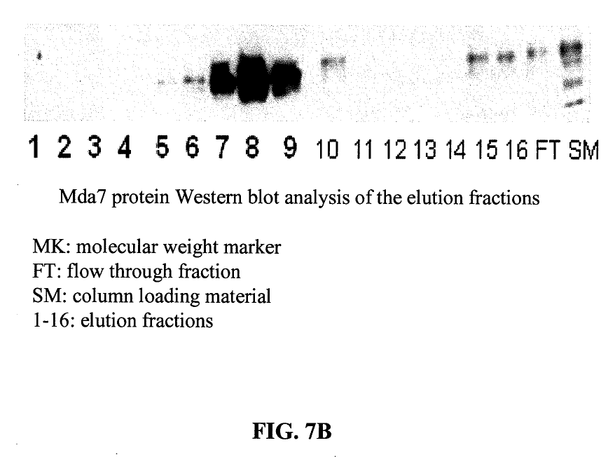

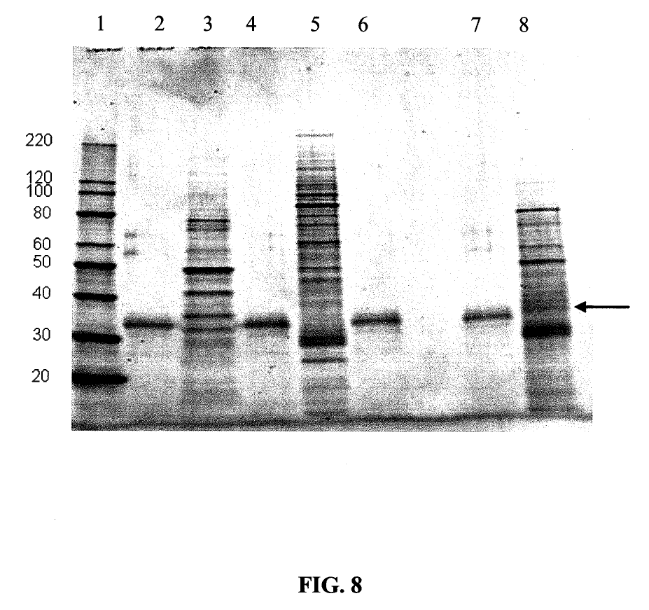

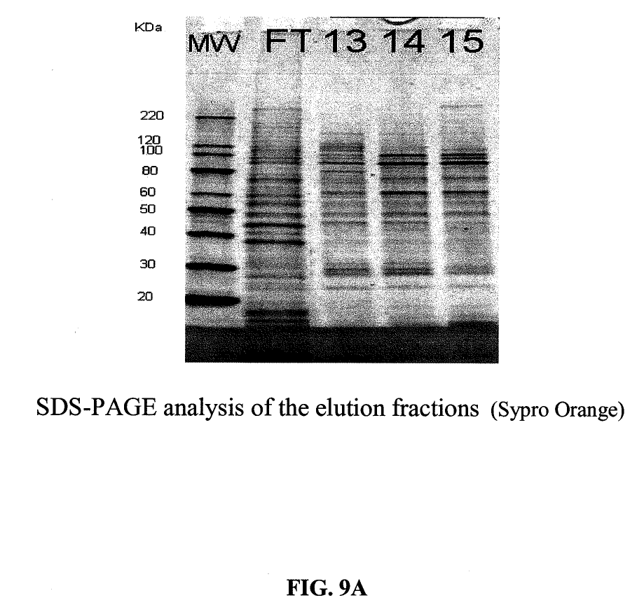

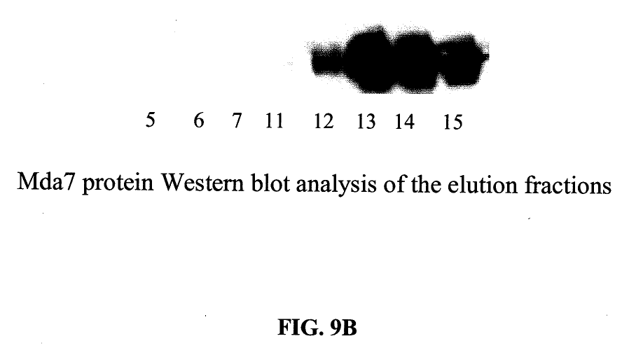

The MDA-7 protein fraction eluted from the phenyl-sepharose FF column was loaded directly onto the equilibrated butyl-sepharose FF column. At the completion of the loading, the column was washed with 5 cv of buffer A to bring the UV absorbance to baseline. A step gradient was used to elute the MDA-7 protein. At the end of the elution, the column was regenerated using the sanitization buffer and stored in 0.01N NaOH solution (FIG. 6) Western blot analysis demonstrated noticeable improvement in mda-7 protein purification (FIG. 7), over phenyl-sepharose FF alone (FIG. 8), or butyl-sepharose FF alone (FIG. 9). The MDA-7 protein band can be seen for the first time on the SDS-PAGE gel (indicated by the arrow on the gel image). Unfortunately, majority of the protein bands still appeared to be impurity proteins.

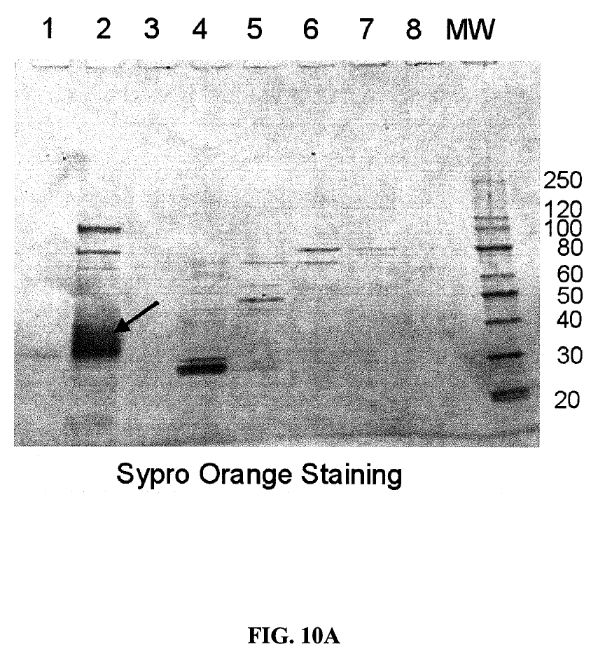

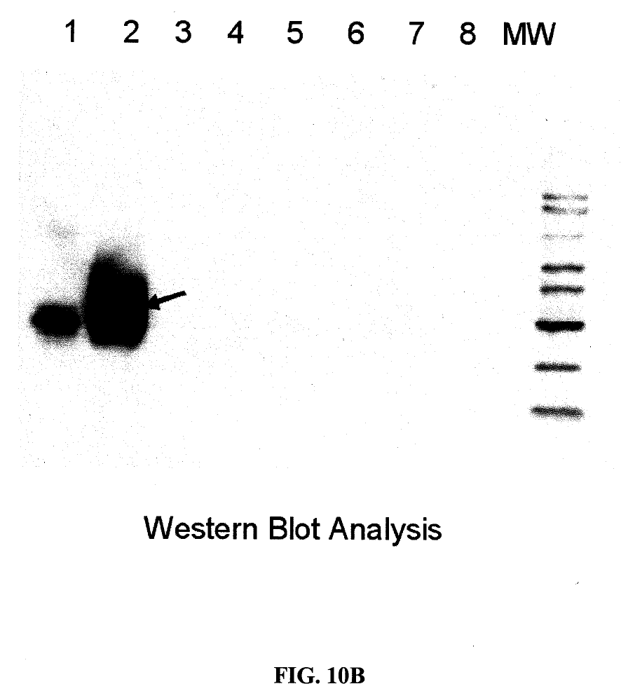

Since there was still a significant amount of impurity proteins present in the MDA-7 protein fraction collected from the butyl-sepharose FF column, further purification was needed. After a number of investigation runs including size exclusion and ion exchange chromatography, satisfactory purification was achieved using the hydroxyapatite type 1 resin. The results showed that MDA-7 protein did not bind to the hydroxyapatite Type 1 column and was collected in the flow through and wash fractions (data not shown). To improve the analysis by SDS-PAGE and MDA-7 Western blot, the MDA-7 flow through fraction was concentrated approximately 300-fold using a 10 kd Centrifree-15 centrifugation concentration device (Cat# UFV2BGC40, Lot# VS1550, Millipore). Following this concentration step, substantially pure Mda-7 protein was obtained after the Hydroxyapatite Type 1 column. An MDA-7 protein band was clearly seen on the SDS-PAGE gel (FIG. 10).

### Example 4

### Optimization of HeLa cell infection condition for Production of MDA-7 Protein

### A. Materials and Methods

1. Cell Lines And Culture

HeLa cells were cultured as described in Example 1.

2. Recombinant Adenovirus

The recombinant adenovirus vectors carrying the mda-7 gene (Ad-mda7) was obtained from Introgen Therapeutics (Introgen Therapeutics, Houston, Tex.). Production of the replication-deficient human type 5 Adenovirus (Ad5) containing the mda-7 gene (Ad-mda7) has been previously reported (Saeki et al., 2000; Mhashilkar, 2001; Pataer et al., 2002). Construction of Ad-mda-7 involved linking mda-7 cDNA to a CMV-IE promoter, followed by an SV40 polyadenylation [p(A)] sequence; this expression cassette was placed in the E1 region of Ad5.

3. Viral Infection

HeLa cells were infected with Ad-mda7 virus at a cell concentration of 1.3×10⁶ cells/ml with varying MOI. MOI was 100, 1000, 2000, or 3000 viral particles (vp)/cell. Samples of the culture media were collected at different time points (3, 4, 5, 6 or 7 days) after virus infection for MDA-7 protein analysis using western blot.

4. Western Blot

Total protein was isolated from the harvested cells by adding cell lysis buffer (20 mM HEPES, pH 7.5; 10 mM KCl, 1 mM MgCl₂, 1 mM EDTA, 1 mM DTT, 250 mM sucrose and 1× protease inhibitor). Proteins were separated by SDS polyacrylamide gel electrophoresis and immobilized on nitrocellulose membranes. Membranes were blocked with 5% nonfat dry milk and incubated overnight at 4° C. with anti MDA-7 (Introgen Therapeutics). Membranes were then washed and incubated with horseradish peroxidase (HRP)-conjugated secondary antibodies for 1 hr at room temperature. Following incubation, the membranes were developed and protein signals detected using enhanced chemilluminescence (ECL) western blotting detection reagents (Amersham Biosciences, Buckinghamshire, UK).

### B. Results

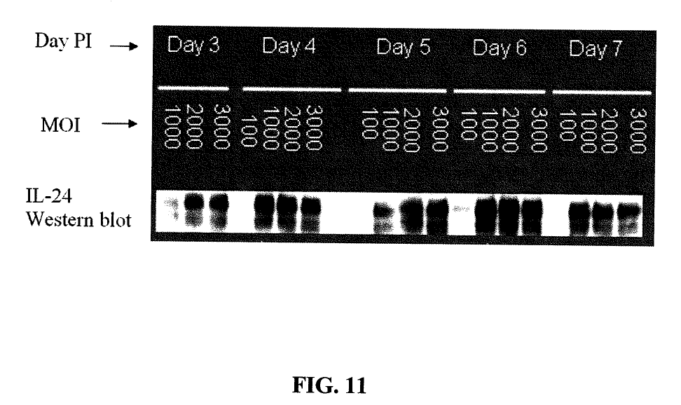

Levels of MDA-7 protein in the culture media increased as the virus infection proceeded. The highest levels of MDA-7 protein were observed after 6 days post infection when HeLa cells were infected with Ad-mda7 at a MOI of 3000 vp/cell. (FIG. 11).

### Example 5

### Comparison of Tumor Cell Killing Activity of Supernatants from AD-mda7 Infected HeLa Cells

### A. Materials and Methods

1. Cell Culture Methodology

HeLa cells were cultured as described in Example 1. Additionally for this example, the MDA-MB-453 breast cancer line, and MeWo, a melanoma cell line, were were obtained from American Type Culture Collection (ATCC, Rockville, Md.). All cells were maintained in Dulbecco's modified Eagle's medium (DMEM, 4.5 g/L glucose) supplemented with 10% fetal bovine serum, 10 mM glutamine, 100 units/ml penicillin, 100 μg/ml streptomycin (Life Technologies, Inc., Grand Island, N.Y.) in a 5% CO₂ atmosphere at 37° C. MDA-MB-453 and MeWO cells were plated in 6-well plates and were subsequently exposed to MDA7 protein produced from Ad-mda-7 infected HeLa cells.

2. Recombinant Adenovirus

The recombinant adenovirus vectors carrying the mda7 gene (Ad-mda7) was obtained from Introgen Therapeutics (Introgen Therapeutics, Houston, Tex.). Details of this vector are described in Example 1.

3. Viral Infection

HeLa cells were infected with Ad-mda7 virus at a cell concentration of 2.30×10⁶ cells/ml in a wave 20 bioreactor with a MOI of 1000 viral particles (vp)/cell. Four days post infection supernatant was harvested and subjected to filtration through a 0.1 μm filter to remove cellular debris.

4. Tumor Cell Killing Assay

Supernatant collected from Ad-mda7 infected HeLa cells was placed on MDA-MB-543 cells or MeWo cells in culture. Volumes of supernatant used to treat MDA-MB-543 cells was 0, 0.1, 0.2, or 0.5 ml of supernatant. As a control, supernatant treated by boiling or treated with MDA-7 antibody was also used (0.1 ml each). Seventy five hours after supernatant exposure, cells were trypsinized and an aliquot was used for staining with 0.4% trypan blue. Total cell numbers and cell viability counts were assessed using a hemocytometer by light microscopy (Chada et al., 2005).

### B. Results

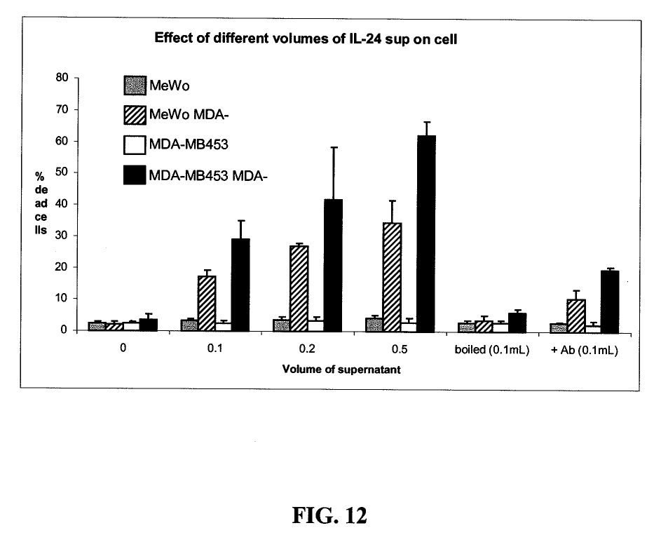

As shown in FIG. 12, Levels of MDA-7 protein in the culture media resulted in dramatic increase in cell death of both MeWo and MDA-MB-543 cell lines as compared to supernatant from HeLa cells which were not infected with Ad-mda7. Also observed was the fact that the percentage of cell death was dose dependent, with increasing dosages of supernatant containing MDA-7 protein resulting in greater levels of target cell death.

All of the compositions and/or methods disclosed and claimed herein can be made and executed without undue experimentation in light of the present disclosure. While the compositions and methods of this invention have been described in terms of some embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### X. REFERENCES

The following references, to the extent that they provide exemplary procedural or other details supplementary to those set forth herein, are specifically incorporated herein by reference.
U.S. Pat. No. 3,917,527
U.S. Pat. No. 4,000,098
U.S. Pat. No. 4,332,717
U.S. Pat. No. 4,352,883
U.S. Pat. No. 4,683,202
U.S. Pat. No. 4,743,680
U.S. Pat. No. 4,771,128
U.S. Pat. No. 4,894,439
U.S. Pat. No. 4,908,434
U.S. Pat. No. 4,920,196
U.S. Pat. No. 5,252,216
U.S. Pat. No. 5,925,565
U.S. Pat. No. 5,928,906
U.S. Pat. No. 5,935,819
Angel et al., Mol. Cell. Biol., 7:2256, 1987.
Angel et al., Cell, 49:729, 1987b.
Atchison and Perry, Cell, 48:121, 1987.
Bates et al., Nature, 395:124-125, 1998.
Banerji et al., Cell, 33(3):729-740, 1983.
Berg et al., BioTechniques, 14(6):972-978, 1993.
Berkhout et al., Cell, 59:273-282, 1989.
Blanar et al., EMBO J, 8:1139, 1989.
Blumberg et al., Cell, 104(1):9-19, 2001.
Bodine and Ley, EMBO J, 6:2997, 1987.
Boshart et al., Cell, 41:521, 1985.
Bosze et al., EMBO J, 5(7):1615-1623, 1986.
Braddock et al., Cell, 58:269, 1989.
Bulla and Siddiqui, J. Virol., 62:1437, 1986
Campere and Tilghman, Genes and Dev., 3:537, 1989.
Campo et al., Nature, 303:77, 1983.
Capaldi et al., Biochem. Biophys. Res. Commun., 74:425-433, 1977.
Carbonelli et al., FEMS Microbiol. Lett., 177(1):75-82, 1999.
Celander and Haseltine, J. Virology, 61:269, 1987.
Celander et al., J. Virology, 62:1314, 1988.
Chandler et al., Cell, 33:489, 1983.
Chang et al., Mol. Cell. Biol., 9:2153, 1989.
Chatterjee et al., Proc. Natl. Acad. Sci. USA, 86:9114, 1989.
Choi et al., Cell, 53:519, 1988.
Cocea, Biotechniques, 23(5):814-816, 1997.
Cohen et al., J. Cell. Physiol., 5:75, 1987.
Costa et al., Mol. Cell. Biol., 8:81, 1988.
Cripe et al., EMBO J, 6:3745, 1987.
Culotta and Hamer, Mol. Cell. Biol., 9:1376, 1989.
Dandolo et al., J. Virology, 47:55-64, 1983.
Deschamps et al., Science, 230:1174-1177, 1985.
De Villiers et al., Nature, 312(5991):242-246, 1984.
Dumoutier et al., Proc. Natl. Acad. Sci. U.S.A., 97:10144-10149, 2000.
Edbrooke et al., Mol. Cell. Biol., 9:1908, 1989.
Edlund et al., Science, 230:912-916, 1985.
Ekmekcioglu et al., Int. J. Cancer, 94(1):54-59, 2001.
Ekmekciouglu et al., Melanoma Res., 9(3):261-272, 1999.
Fausnaugh, J. L. and F. E. Regnier, J. Chromatog., 359:131-146, 1986.
Feng and Holland, Nature, 334:6178, 1988.
Firak and Subramanian, Mol. Cell. Biol., 6:3667, 1986.
Foecking and Hofstetter, Gene, 45(1):101-105, 1986.
Fujita et al., Cell, 49:357, 1987.
Gallagher et al., Genes Immun., 1(7):442-450, 2000.
Garnier et al., Cytotechnology, 15:145-155, 1994.
Ghosh-Choudhury et al., EMBO J, 6:1733-1739, 1987.
Gilbert, “Adaptation of cells to serum free culture for production of adenovirus vectors and recombinant proteins,” Williamsburg BioProcessing Conference, Nov. 18-21, 1996.
Gilles et al., Cell, 33:717, 1983.
Gloss et al., EMBO J., 6:3735, 1987.
Godbout et al., Mol. Cell. Biol., 8:1169, 1988.
Gomez-Foix et al., J. Biol. Chem., 267:25129-25134, 1992.
Goodbourn and Maniatis, Proc. Natl. Acad. Sci. USA, 85:1447, 1988.
Goodboum et al., Cell, 45:601, 1986.
Graham and Prevec, In: Methods in Molecular Biology: Gene Transfer and Expression Protocol, Murray (Ed.), Humana Press, NJ, 7:109-128, 1991.
Graham et al, J. Gen. Virl., 36(1):59-74, 1977.
Greene et al., Immunology Today, 10:272, 1989
Griffiths, J. B., In “Animal Cell Biotechnology”, vol. 3, p179-220, (Eds. Spier, R. E. and Griffiths, J. B.), Academic Press, London., 1986
Grosschedl and Baltimore, Cell, 41:885, 1985.
Grunhaus and Horwitz, Seminar in Virology, 3:237-252, 1992.
Haslinger and Karin, Proc. Natl. Acad. Sci. USA, 82:8572, 1985.
Hauber and Cullen, J. Virology, 62:673, 1988.
Hauber and Cullen, J. Virology, 62:673, 1988.
Hensel et al., Lymphokine Res., 8:347, 1989.
Herr and Clarke, Cell, 45:461, 1986.
Hirochika et al., J. Virol., 61:2599, 1987.
Hirsch et al., Mol. Cell. Biol., 10:1959, 1990.
Holbrook et al., Virology, 157:211, 1987.
Horlick and Benfield, Mol. Cell. Biol., 9:2396, 1989.
Huang et al., Cell, 27:245, 1981.
Hug et al., Mol. Cell. Biol., 8:3065, 1988.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Jiang et al., Nature, 375:151-155, 1995.
Jiang et al., Oncogene, 11(12):2477-2486, 1995.
Johnson et al., Mol. Cell. Biol., 9:3393, 1989.
Jones and Shenk, Cell, 13:181-188, 1978.
Kadesch and Berg, Mol. Cell. Biol., 6:2593, 1986.
Karin et al., Mol. Cell. Biol., 7:606, 1987.
Karlsson et al., EMBO J., 5:2377-2385, 1986.
Katinka et al., Cell, 20:393, 1980.
Kawamoto et al., Mol. Cell. Biol., 8:267, 1988.
Kiledjian et al., Mol. Cell. Biol., 8:145, 1988.
Klamut et al., Mol. Cell. Biol., 10:193, 1990.
Knapp et al., Atherosclerosis, 152(1):217-227, 2000.
Koch et al., Mol. Cell. Biol., 9:303, 1989.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kuhl et al., Cell, 50:1057, 1987.
Kunz et al., Nucl. Acids Res., 17:1121, 1989.
Larsen et al., Proc Natl. Acad. Sci. USA., 83:8283, 1986.
Laspia et al., Cell, 59:283, 1989.
Latimer et al., Mol. Cell. Biol., 10:760, 1990.
Lee et al., Nature, 294:228, 1981.
Levenson et al., Hum. Gene Ther., 9(8):1233-1236, 1998.
Levinson et al., Nature, 295:79, 1982.
Levrero et al., Gene, 101: 195-202, 1991.
Lin et al., Mol. Cell. Biol., 10:850, 1990.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Majors and Varmus, Proc. Natl. Acad. Sci. USA, 80:5866, 1983.
McNeall et al., Gene, 76:81, 1989.
Mhashilkar et al., Mol. Med., 7:271-282, 2001.
Miksicek et al., Cell, 46:203, 1986.
Mizrahi, Process Biochem., (August):9-12, 1983.
Mordacq and Linzer, Genes and Dev., 3:760, 1989.
Moreau et al., Nucl. Acids Res., 9:6047, 1981.
Muesing et al., Cell, 48:691, 1987.
Ng et al., Nucl. Acids Res., 17:601, 1989.
Ondek et al., EMBO J., 6:1017, 1987.
Ornitz et al., Mol. Cell. Biol., 7:3466, 1987.
Pataer et al., Cancer Res., 62:2239-2243, 2002.
Palmiter et al., Nature, 300:611, 1982.
Pech et al., Mol. Cell. Biol., 9:396, 1989.
Perez-Stable and Constantini, Mol. Cell. Biol., 10:1116, 1990.
Perrin et al., Vaccine, 13(13):1244-1250, 1995.
Phillips et al., In: Large Scale Mammalian Cell Culture (Feder, J. and Tolbert, W. R., eds.), Academic Press, Orlando, Fla., U.S.A., 1985.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Queen and Baltimore, Cell, 35:741, 1983.
Quinn et al., Mol. Cell. Biol., 9:4713, 1989.
Redondo et al., Science, 247:1225, 1990.
Reisman and Rotter, Mol. Cell. Biol., 9:3571, 1989.
Renan, Radiother. Oncol., 19:197-218, 1990.
Resendez Jr. et al., Mol. Cell. Biol., 8:4579, 1988.
Ripe et al., Mol. Cell. Biol., 9:2224, 1989.
Rittling et al., Nuc. Acids Res., 17:1619, 1989.
Rosen et al., Cell, 41:813, 1988.
Saeki et al., Gene Ther., 7:2051-2057, 2000.
Sakai et al., Genes and Dev., 2:1144, 1988.
Sambrook et al., In. Molecular cloning, Cold Spring Harbor Laboratory Press, Cold Spring Harbor, N.Y., 2001.
Satake et al., J. Virology, 62:970, 1988.
Schaefer et al., Cell Signal, 12(3):143-151, 2000.
Schaffner et al., J. Mol. Biol., 201:81, 1988.
Searle et al., Mol. Cell. Biol., 5:1480, 1985.
Sharp and Marciniak, Cell, 59:229, 1989.
Shaul and Ben-Levy, EMBO J., 6:1913, 1987.
Sherman et al., Mol. Cell. Biol., 9:50, 1989.
Sleigh and Lockett, J. EMBO, 4:3831, 1985.
Soo et al., J. Cell. Biochem., 74(1):1-10, 1999.
Spalholz et al., Cell, 42:183, 1985.
Sullivan and Peterlin, Mol. Cell. Biol., 7:3315, 1987.
Spandidos and Wilkie, EMBO J., 2:1193, 1983.
Spandau and Lee, J. Virology, 62:427, 1988.
Stephens and Hentschel, Biochem. J, 248:1, 1987.
Stuart et al., Nature, 317:828, 1985.
Swartzendruber and Lehman, J. Cell. Physiology, 85:179, 1975.
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10: 176, 1990b.
Taylor et al., J. Biol. Chem., 264:15160, 1989.
Thiesen et al., J. Virology, 62:614, 1988.
Tronche et al., Mol. Biol. Med., 7:173, 1990.
Trudel and Constantini, Genes and Dev., 6:954, 1987.
Tyndell et al., Nuc. Acids. Res., 9:6231, 1981.
Vannice and Levinson, J. Virology, 62:1305, 1988.
Vasseur et al., Proc Natl. Acad. Sci. USA, 77:1068, 1980.
Vogelstein et al., Nature, 408(6810):307-310, 2000.
Wang and Calame, Cell, 47:241, 1986.
Weber et al., Cell, 36:983, 1984.
Weinberger et al. Mol. Cell. Biol., 8:988, 1984.
Winoto and Baltimore, Cell, 59:649, 1989.
Yutzey et al. Mol. Cell. Biol., 9:1397, 1989.
Zhang et al., Endocrinology, 141:4698-4710, 2000.

What is claimed is:

1. A method for producing an exogenous protein comprising the steps of:
(i) infecting non-trans-complementing host cells capable of exogenous protein expression with a replication-defective adenoviral vector comprising a nucleic acid expression construct with a nucleic acid sequence encoding one or more exogenous proteins;
(ii) growing the infected cells; and
(iii) harvesting the exogenous protein or proteins produced by the non trans-complementing host cell from a cell extract or supernatant.

2. The method of claim 1, wherein the non-trans-complementing host cells are Vero, HeLa, Chinese hamster ovary, W138, BHK, COS-7, HepG2, RIN, MDCK, A549 or derivatives thereof.

3. The method of claim 1, wherein the non-trans-complementing host cells are adapted for growth in serum-free media.

4. The method of claim 2, wherein the non-trans-complementing host cells are HeLa or a derivative thereof.

5. The method of claim 1, wherein the adenovirus vector has a mutation in the E1 region of the virus.

6. The method of claim 5, wherein the mutation is a deletion of all or part of the E1 region.

7. The method of claim 5, wherein the adenovirus vector further has a mutation in the E3 region of the virus.

8. The method of claim 7, wherein the mutation is a deletion of all or part of the E3 region.

9. The method of claim 1, wherein the nucleic acid expression construct further comprises one or more heterologous promoters.

10. The method of claim 9, wherein the promoter or promoters are selected from the group consisting of constitutive promoters, inducible promoters or tissue selective promoters.

11. The method of claim 10, wherein said promoter or promoters are selected from the group of CMV IE promoter, dectin-1 promoter, dectin-2 promoter, human CD11c promoter, mammalian F4/80 promoter, SM22a promoter, MHC class II promoter, hTERT promoter, CEA promoter, PSA promoter, probasin promoter, ARR2PB promoter, AFP promoter, SV40 early promoter, the U3 region of the Rous sarcoma virus, the U3 region of Mason-Pfizer monkey virus, and any inducible promoter capable of operating in mammalian cells.

12. The method of claim 1, wherein the nucleic acid expression construct further comprises one or more heterologous polyadenylation signals.

13. The method of claim 12, wherein said polyadenylation signal or signals are selected from the group of SV40 early polyadenylation signal, HSV TK polyadenylation signal, and human growth hormone polyadenylation signal.

14. The method of claim 1, wherein the infected cells are grown in suspension in serum-free media.

15. The method of claim 1, wherein the infected cells are grown in media that lacks protein.

16. The method of claim 1, further comprising purifying the harvested exogenous protein or proteins.

17. The method of claim 16, wherein purifying the exogenous protein or proteins involves chromatography.

18. The method of claim 17, wherein the chromatography is affinity chromatography, hydrophobic interaction chromatography, hydroxyapatite and/or ion chromatography.

19. The method of claim 18, wherein the ion chromatography is anion exchange chromatography.

20. The method of claim 18, wherein the hydrophobic interaction chromatography involves phenyl-sepharose chromatography.

21. The method of claim 18, wherein the hydrophobic interaction chromatography involves butyl-sepharose chromatography.

22. The method of claim 21, further comprising hydroxyapatite chromatography.

23. The method of claim 18, wherein the heterologous protein or proteins are purified using affinity chromatography and anion exchange chromatography.

24. The method of claim 16, wherein purifying the exogenous protein or proteins comprises subjecting the protein or proteins to size resolution purification.

25. The method of claim 24, wherein size resolution purification involves a protein gel or a size exclusion column.

26. The method of claim 16, wherein the purified exogenous protein or proteins is formulated in a pharmaceutically acceptable composition.

27. The method of claim 1, wherein the protein is selected from the group consisting of tumor suppressors, cytokines, antibodies and pro-apoptotic factors.

28. The method of claim 27, wherein the tumor suppressor is selected from the group consisting of APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MDA-7, MEN1, MEN2, MTS1, NF1, NF2, VHL, WRN, WT1, CFTR, C-CAM, CTS-1, zac1, ras, MMAC1, FCC, MCC, FUS1, Gene 26 (CACNA2D2), PL6, Beta* (BLU), Luca-1 (HYAL1), Luca-2 (HYAL2), 123F2 (RASSF1), 10F6, Gene 21 (NPRL2), or a gene encoding a SEM A3 polypeptide.

29. The method of claim 27, wherein the wherein the cytokine is selected from the group consisting of GM-CSF, G-CSF, IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32 IFN-α, IFN-β, IFN-γ, MIP-1α, MIP-1p, TGF-β, TNF-α, TNF-β, PDGF, epidermal growth factor, keratinocyte growth factor, hepatycyte growth factor, TGF-α, TGF-β, VEGF and mda-7.

30. The method of claim 27, wherein the wherein the pro-apoptotic factor is selected from the group consisting of CD95, caspase-3, Bax, Bag-1, CRADD, TSSC3, bax, hid, Bak, MKP-7, PARP, bad, bcl-2, MST1, bbc3, Sax, BIK, and BID.

31. The method of claim 27, wherein the antibody is selected from the group consisting of cetuximab, rituximab, trastuzumab, gemtuzumab, alemtuzumab, ibritumomab, tositumomab, bevacizumab, alemtuzumab, HuPAM4, 3F8, G250, HuHMFG1, Hu3S193, hA20, SGN-30, RAV12, daclizumab, basiliximab, abciximab, palivizumab, infliximab, eculizumab, omalizumab, efalizumab, and adalimumab.

32. The method of claim 1, wherein the protein is from an organisms selected from the group consisting of viruses, bacteria, fungi, and protozoa.

33. The method of claim 32, wherein the microorganisms are viruses selected from a list of HIV-1, HIV-2, SIV, FIV, FeLV, Equine infectious anemia virus, eastern equine encephalitis virus, western equine encephalitis virus, Venezuelan equine encephalitis virus, rift valley fever virus, West Nile virus, yellow fever virus, Crimean-Congo hemorrhagic fever virus, dengue virus, SARS coronavirus, small pox virus, monkey pox virus, hepatitis A virus, hepatitis B virus, hepatitis C virus, influenza virus and rotavirus.

34. The method of claim 32, wherein the microorganisms are bacteria selected from a list of Mycobacterium tuberculosis, Yersinia pestis, Rickettsia prowazekii, Rickettsia typhi, Rickettsia rickettsii, Ehrlichia chaffeensis, Rrancisella tularensis, Bacillus anthracis, Helicobacter pylori and Borrelia burgdorferi.

35. The method of claim 32, wherein the microorganisms are protozoa selected from a list of Plasmodium falciparum, Plasmodium vivax, Plasmodium ovale, Plasmodium malariae and Giadaria intestinalis.

36. The method of claim 32, wherein the microorganisms are fungi selected from a list of Histoplasma, Ciccidis, Immitis, Aspergillus, Actinomyces, Blastomyces, Candida and Streptomyces.

37. The method of claim 1, wherein infecting the culture of non trans-complementing host cells occurs in a bioreactor system, a microcarrier culture system, a multiplate culture system, a perfused packed bed reactor system, or a microencapsulation culture system.

38. A method for producing an exogenous protein comprising:
(i) infecting non-trans-complementing host cells capable of exogenous protein expression with a replication-defective adenoviral vector comprising a nucleic acid expression construct with a nucleic acid sequence encoding one or more exogenous proteins, wherein the replication adenoviral vector is mutated in the E1 region;
(ii) growing the infected cells in serum-free media;
(iii) harvesting the heterologous protein or proteins produced by the non-trans-complementing host cell from a cell extract or supernatant; and,
(iv) purifying the exogenous protein or proteins.
